(19) United States
(12) Patent Application Publication
(10) Pub. No.: US 2026/0151599 A1
(43) Pub. Date: Jun. 4, 2026

(54) STEERABLE RIGIDIZING CATHETER SYSTEMS AND METHODS

(71) Applicant: JUPITER ENDOVASCULAR INC., Menlo Park, CA (US)

(72) Inventors: Benjamin Y. HU, Cary, NC (US); Garrett J. GOMES, Pleasant Hill, CA (US); Jeffrey NORTON, Emerald Hills, CA (US); Alexander Q. TILSON, Burlingame, CA (US)

(21) Appl. No.: 19/405,377

(22) Filed: Dec. 1, 2025

Related U.S. Application Data
(60) Provisional application No. 63/728,143, filed on Dec. 4, 2024.

(51) Int. Cl. A61M 25/01; A61B 17/00; A61B 17/22; A61B 90/00; A61M 25/00

(52) U.S. Cl. CPC A61M 25/0155; A61B 17/22; A61B 90/06; A61M 25/0045; A61M 25/0102; A61B 1/00078; A61B 1/00135; A61B 1/015; A61B 2017/00022; A61B 2017/00544; A61B 2017/00557; A61B 2017/00778; A61B 2017/22051; A61B 2017/22079; A61B 2090/064; A61B 2217/005; A61M 25/0043; A61M 2025/0063; A61M 2202/0014; A61M 2202/06; A61M 2205/3344; A61M 2210/12

(57) ABSTRACT

Steerable rigidizing catheter systems and methods. A rigidizing catheter may be configured to be rigidized upon the introduction of positive or negative pressure within walls of the rigidizing catheter by a pressurizing device. The pressurizing device may be configured to provide refined control of fluid flow into and/or out of the rigidizing catheter such that pressure within the rigidizing catheter can be gradually changed. Gradually changing the pressure may cause at least a distal end region of the catheter to change shape as desired, thereby allowing steering of the catheter's distal end region. The methods are well suited for positioning clot aspiration devices within a subject's vasculature.

### CROSS-REFERENCES TO RELATED APPLICATIONS

The present application claims the benefit of and priority to U.S. Provisional Patent Application No. 63/728,143, filed Dec. 4, 2024, the content of which is hereby incorporated by reference in its entity.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Medical procedures performed within the vasculature often involve navigating through long and tortuous anatomy to reach a site of interest. This complicated navigation and resulting pathway to a treatment site requires highly flexible catheters, which typically results in poor control of the interventional or medical device once treatment is attempted at the destination. There are different optimal stiffnesses for the different activities of navigation and treatment. Heretofore, most catheter designers have tried to solve the problem of balancing stiffness for navigation and for treatment by creating devices whose stiffness is a compromise between those different activities.

Most transcatheter procedures rely on the use of a pre-placed guidewire to direct and navigate the catheter to the desired anatomy. For example, prior to deploying a stent in the coronary artery, a guidewire is advanced through the lesion to serve as a guide for the catheter carrying the stent. However, the use of guidewires may complicate these procedures, requiring extra steps, and may not provide appropriate stability and support when performing operations within the vasculature. Guidewires may also displace the catheter being guided, particularly when navigating through tortuous anatomy. Guidewires may need to be ‘swapped’ for sequentially different stiffness guidewires, which adds time, risk and cost. Guidewire displacement, including losing guidewire position, can affect the ability to place the catheter in the correct position and can significantly extend procedural difficulty, risk, and duration and may lead to complications and iatrogenic injury.

Moreover, guidewires and guide catheters are often not effective for large bore catheters (e.g., large bore catheters suitable for treating pulmonary embolisms) because the large bore catheter may not easily track over the guidewire through tortuous anatomy (e.g., through the right atrium, the tricuspid valve, the right ventricle, the pulmonary valve, and through the pulmonary bifurcations). These larger bore catheters can be quite stiff, which, when navigated amongst tortuous anatomy, create stored energy which can be released unpredictably, causing procedural uncertainty and potentially dangerous complications including iatrogenic injury. In general, stiffer catheters require stiffer guidewires, which have more clinical risks—including perforations—as compared to more flexible guidewires.

A catheter that can be placed in the anatomy in a flexible condition may be better able to conform to the anatomy. Once rigidized, the anatomical pathway is preserved, protecting the adjacent anatomy by limiting forces that straighten the access. In one example, a pathway developed by an access catheter may begin in a state of anatomical conformation, but subsequent instruments placed within the access catheter may increase the stiffness and straighten the sheath and adjacent anatomy. Isolating and preserving the anatomical pathway reduces the potential for iatrogenic injury and hemodynamic complications associated with impingement of certain anatomical structures like heart valves.

Vascular obstructions (clots) may be removed with multiple devices, including mechanical removal, hydraulics (pressurized fluid streams) and by aspiration. Aspiration systems apply vacuum along a lumen from a proximal low-pressure source (for example, a vacuum pump or syringe) to a distal opening. In some cases, vascular obstructions reside in complex and delicate blood vessels, making it challenging for aspiration devices to navigate such narrow, winding pathways without damaging the vessel walls or surrounding tissue.

Furthermore, contemporary aspiration catheters designed for clot removal must exhibit rigidity along most of their length to ensure effective navigation through vascular pathways to prevent buckling and/or undesirable bending, even when used over a guidewire. This rigidity is crucial for preventing catheter collapse under the negative pressures required to aspirate clots, particularly in large vessel occlusions. At the same time, this structural integrity enables the catheter to transmit force effectively during insertion and maneuvering. However, while the majority of the catheter remains rigid for stability, the distal tip is typically more flexible to minimize trauma to the vessel wall and enhance navigation through tortuous anatomy. Balancing rigidity and flexibility is a critical aspect of the design to optimize performance and safety during endovascular procedures. As a result, such catheters typically can only be highly flexible over a relatively short distal region and transition between different stiffnesses gradually over their length.

There is a need for methods and apparatuses that may provide a safer and more reliable pathway to internal treatment sites, and that may allow enhanced navigation through different parts of the vasculature.

### SUMMARY OF THE DISCLOSURE

Described herein are methods and apparatuses (e.g., devices, systems and assemblies), including systems and devices, and in particular catheters and associated components, which may be used to provide access to complex or delicate body lumens and cavities, including in particular, to remove material (e.g., clot material) from a blood vessel. For example, described herein are rigidizing catheters that may be adapted for use within a subject's vasculature (e.g., blood vessels, heart, etc.). As used herein, vasculature may include any vascular region of the body, including, but not limited, to peripheral, neurovascular, etc.

These apparatuses, e.g., rigidizing catheters, may be configured to be rigidized upon application of fluid pressure within walls of the catheters. The fluid pressure may be positive or negative (vacuum). The fluid pressure may be positive in a portion of the wall and negative in another portion of the wall. The fluid may include gas (e.g., air) or liquid (e.g., saline solution). The rigidizing catheters, and in particular the distal end regions of the rigidizing catheters, may be configured to be steered by controlling the timing and rate of pressurization. The controlled change in pressure allows for gradual relaxation and/or rigidization of the catheter. The systems may include a pressurization apparatus that is configured to supply the fluid pressure in a gradual manner such that, for example, the distal end region of the rigidizing catheter can controllably take on a desired shape. The systems provide doctors more control in navigating the catheter through complex anatomical channels, such as winding and/or narrow blood vessels.

The rigidizing catheters described herein may be converted between one or more flexible states that may be readily navigated through even a tortuous anatomy, and one or more more-rigid states, in which the shape (including any bends or curves) of the rigidizing catheter is locked in position. Intermediate states may also be useful at certain times during the procedure. As used herein, the flexible state may be highly flexible and the rigid state is generally more rigid than the flexible state; in some examples the rigid state may be highly rigid. In some cases, the rigidizing catheter may change shape as it transitions between rigid and flexible states, or vice versa. Methods described herein make use of this shape change to steer the rigidizing catheter within the vasculature, especially at a distal end region of the rigidizing catheter.

The apparatuses described herein may address the problem of balancing different stiffnesses for navigation and for treatment by configuring and using devices or systems that may dynamically modulate their stiffnesses (‘dynamically rigidizing’ or ‘dynamically stabilizing’), from high flexibility to high stiffness, throughout the procedure, potentially as a function of location along the device shaft, with a fast, facile, and indefinitely large number of transitions between these variable stiffness states. As mentioned above, these apparatuses may have significant advantages as compared with standard catheters that are not dynamically rigidizing, and typically have a set, fixed stiffness, though those fixed stiffness values can vary by zone (typically the zones change along the catheter's length). The dynamically rigidizing apparatuses (including but not limited to dynamically rigidizing catheters and other components) described herein can have regions that are selectively rigidized, or the entire shaft can be rigidized or made flexible as one cohesive entity. A dynamically rigidizing catheter can be configured to have a substantially lower stiffness over its entire length in a flexible configuration than a typical catheter, which can be advantageous as the proximal zone navigates through anatomy that can be tortuous, calcific, and, in some instances, can extend through atrium, ventricles, and valves. In these instances, lower baseline stiffness puts lower load on localized anatomical features. These same rigidizing catheters may then convert, without substantially foreshortening or shifting position, into a rigid configuration that is sufficiently rigid (e.g. has an effective stiffness along its length) to hold the catheter in a shape that avoids damage to the anatomy through which it has been navigated, even when passing a second catheter or tool that may tend to push against the rigidizing catheter. Controlling the effective stiffnesses may be particularly important within the vasculature in both the rigid and the flexible configuration. In the flexible configuration it is important that the catheter be sufficiently flexible along nearly all of its length so that it does not damage the vasculature (vessels, heart, etc.) when being inserted through the body, while still tracking over a guidewire. In the rigid configuration, it is important that the catheter be sufficiently rigid so that other devices (e.g., aspiration catheters, tools, etc.) passed through the rigidizing catheter cannot deform or displace the rigidizing catheter and potentially harm the vasculature and/or change the position of the distal end region of the catheter.

The rigidizing catheters described herein may be configured as aspiration sheath catheters for applying aspiration (e.g., suction) directly through the lumen of the rigidizing catheter and/or they may be configured to receive an aspiration catheter (which, in some examples, may or may not also be rigidizing). A proximal end of the rigidizing catheters described herein may be adapted for locking or coupling to a distal end of a vacuum line to provide the aspiration force. In some cases, an aspiration catheter (or other device) may be inserted into the lumen of the rigidizing catheter while still allowing aspiration to be applied through the lumen of the rigidizing catheters. For example, the apparatus may include an aspiration line that engages with the proximal end of the rigidizing catheter including a hemostasis valve to allow insertion (and form a seal around the inserted) aspiration catheter or other device.

In general, the methods and apparatuses described herein are particularly useful for vascular indications, including cardiovascular, peripheral vascular, cerebrovascular, neurovascular, pulmonary vascular, thoracic vascular, abdominal vascular, lymphatic vascular, renal vascular, and/or genitourinary vascular. The rigidizing catheter may be dynamically rigidized, e.g., switched between a rigid state and a flexible state. The rigidizing catheter may also include a hemostatic valve region at the proximal end. The hemostatic valve region may be integrated into the rigidizing catheter. In some examples the rigidizing catheter may be rigidized using positive pressure; alternatively in some examples the rigidizing catheter may be rigidized using negative pressure. In some examples the rigidizing catheter may be rigidized by both negative and positive pressure.

Methods described herein may include positioning a rigidizing device in a region of a patient's body so that a distal end region of the rigidizing device is maintained in a rigid configuration that is bent or curved into a first shape or configuration by holding pressure within the rigidizing device at a first pressure; changing the pressure in the rigidizing device to a second pressure until the rigidizing device gradually relaxes into a secondary shape or configuration; monitoring movement of the distal end region of the rigidizing device as the pressure is changed; and rigidizing the rigidizing device once the distal end region has achieved a desired secondary position by re-applying pressure. This allows for precise placement of the distal end region of the rigidizing device, thereby allowing access to channels or cavities that are otherwise not accessible. In some cases, this may involve steering the distal end region of the rigidizing catheter through a branched blood vessel region of the vasculature.

For example, described herein are methods of steering a rigidizing device within a body by adjusting (e.g., dynamically adjusting) a rigidity of the rigidizing device. For example, the method may include: maintaining the rigidizing device in a rigid state having a first configuration in a first region of the body by holding a pressure within the rigidizing device at a first pressure; changing the pressure within the rigidizing device from the first pressure to a second pressure at which the rigidizing device is more flexible than the rigid state, so that the rigidizing device relaxes from the first configuration towards a second configuration; monitoring movement of the rigidizing device as the rigidizing device relaxes towards the second configuration in a second region of the body; and rigidizing the rigidizing device by re-applying pressure once the rigidizing device is repositioned within the body in a desired position.

The rigidizing device may track over a guidewire but it may also track over an obturator or dilator, including rigidizing or steerable versions. A rigidizing obturator or dilator may also be referred to as a DRG or Dynamically Rigidizing Guiderail, including as described in PCTUS2021049165, titled “DYNAMICALLY RIGIDIZING GUIDERAIL AND METHODS OF USE.”

In general, the methods and apparatuses described herein may provide superior control of the catheter or catheters within the vasculature by using dynamic rigidization. For example, a rigidizing catheter may be positioned at or near a target location within the body in the flexible configuration (or by shifting between a rigid and flexible state), and then may be rigidized to provide stability to other components that may pass through and/or extend from the apparatus. These methods and apparatuses may be used with a guidewire, e.g., to help position and/or navigate the catheter, however it may be preferable to operate these apparatuses without the use of a guidewire. Thus, these methods and apparatuses may be used without a guidewire (or in some cases without re-introducing a guidewire), as the position of the catheter may be maintained by rigidizing the rigidizing aspiration sheath catheter. Thus, the rigidizing catheter may be both a conduit for delivery and/or removal of material within the vasculature and may provide a stable platform for performing all or part of the procedure, including clot material and further navigation.

Any of these methods may repeat the processes of changing the pressure, monitoring movement and rigidizing the rigidizing device to steer the rigidizing device within the body to the second configuration. These steps may be repeated (together or individually) to position the distal end of the catheter. Re-applying the pressure may comprise applying pressure from the second pressure back towards the first pressure. In any of these examples, holding the pressure within the rigidizing device at the first pressure may comprise holding the pressure against a bladder layer so that the bladder layer within a wall of the rigidizing device drives a rigidizing layer comprising a plurality of filaments against a reinforced layer of the rigidizing device. Changing the pressure within the rigidizing device may comprise leaking the pressure gradually at a rate that is less than the rate that pressure is re-applied to rigidizing the rigidizing device. For example, leaking the pressure may comprise changing the pressure at a rate of 50 mmHg/sec or less. The first configuration may comprise a bent or curved configuration; the second configuration may be set by an elongate member inserted through the rigidizing device. For example, the elongate member may comprise an obturator. The second configuration may be set by a shape set on the rigidizing device in the flexible state. In any of these apparatuses, holding the pressure may comprise holding a positive pressure. For example, holding the pressure may comprise holding a negative pressure. The body may comprise a branched region within a subject's vasculature.

Any of these methods may include positioning the rigidizing device within the body while the rigidizing device is in the flexible configuration, prior to maintaining the rigidizing device in the rigid state. The second configuration of the rigidizing device in the flexible state may be a straight configuration. In general, changing the pressure may comprise releasing the pressure to atmosphere. The pressure may be a hydrostatic pressure.

For example, a method of positioning a distal tip region of a rigidizing catheter within a body may include: positioning the distal tip region of the rigidizing catheter towards a first vessel while the rigidizing catheter is in a flexible state; rigidizing the rigidizing catheter from the flexible state to a more rigid state by applying pressure to the rigidizing catheter to hold the distal tip region of the rigidizing catheter oriented towards the first vessel; changing the pressure in the rigidizing catheter to relax the rigidizing catheter from the rigid state in which the distal tip region of the rigidizing catheter is oriented towards the first vessel into a flexible configuration so that the distal tip region of the rigidizing catheter moves towards a second vessel; and rigidizing the rigidizing catheter once the distal tip region is oriented towards the second vessel by re-applying pressure within the rigidizing catheter.

Any of these methods may include performing a first procedure with the distal tip region oriented towards the first vessel when the rigidizing catheter is in the rigid configuration and performing a second procedure with the distal tip region oriented towards the second vessel when the rigidizing catheter is in the rigid configuration. For example, the first and second procedures may comprise removing clot material. Applying pressure may comprise applying positive or negative pressure within a wall of the rigidizing catheter. In some cases applying pressure comprises applying positive or negative pressure between one or more layers of the rigidizing catheter. Changing the pressure may comprise slowly releasing the pressure. For example, changing the pressure comprises repeating the steps of releasing the pressure and re-applying the pressure to orient the distal tip region towards the second vessel before rigidizing the rigidizing catheter once the distal tip region is oriented towards the second vessel.

Also described herein are apparatuses (e.g., devices, systems, etc. including hardware, firmware and/or software for performing any of the techniques described herein). For example, described herein are apparatuses comprising: a rigidizing catheter that is configured to transition between a flexible state to a rigid state based on the application of pressure within a wall of the rigidizing catheter; and a pressure controller that is configured to apply or remove a fluid to control the pressure within the wall of the rigidizing catheter, wherein the pressure controller comprises a first input (e.g., a button, lever, position on input etc.) configured to release pressure from the wall of the rigidizing catheter at a first rate, and a second input configured to release pressure from the wall of the rigidizing catheter at a second rate that is less than the first rate to provide a gradual transition between the rigid and flexible states of the rigidizing catheter.

The second input may be configured to release pressure from the wall of the rigidizing catheter by a predefined rate (e.g., a rate of 10 mmHg/sec or less, 20 mmHg/sec or less, 30 mmHg/sec or less, 40 mmHg/sec or less, 50 mmHg/sec or less, 60 mmHg/sec or less, 70 mmHg/sec or less, 80 mmHg/sec or less, 90 mmHg/sec or less, between about 10-100 mmHg/sec, between about 20 mmHg/sec and 80 mmHg/sec, etc.). The pressure controller may include a pressure sensor that is configured to detect the pressure with rigidizing catheter. Other sensors may also or alternatively be used (e.g., flow sensor, etc.).

In any of these apparatuses, the rigidizing catheter may be configured to have a bias shape in the flexible state. For example, the bias shape may be straight, curved, bent, etc. particularly at the distal end region (e.g., the distal-most x mm or more, such as distal-most 5 mm, distal-most 10 mm, distal-most 12 mm, distal-most 15 mm, distal-most 20 mm, distal-most 25 mm, distal most 30 mm, distal-most 35 mm, distal-most 40 mm, distal-most 45 mm, distal-most 50 mm, distal-most 55 mm, distal-most 60 mm, distal-most 70 mm, distal-most 80 mm, distal-most 90 mm, distal-most 100 mm, etc.). In some cases the bias shape is curved. In some cases the bias may be bent or angled. In some cases the bias shape is L-shaped or shaped like a hockey stick. The bias may be relatively weak. For example, the bias shape can be overcome by less than 0.05 N force (e.g., 0.10 N or less, 0.09 N or less, 0.08 N or less, 0.07 N or less, 0.06 N or less, 0.05 N or less, 0.04 N or less, 0.03 N or less, 0.02 N or less, 0.01 N or less, etc.).

Any of these apparatuses (systems) may include an aspiration catheter configured to be inserted into a lumen of the rigidizing catheter and/or an obturator. The obturator may be steerable. The obturator may be rigidizing (‘DRG’, or Dynamically Rigidizing Guiderail). The obturator may have an effective stiffness that is less than or equal to the effective stiffness of the rigidizing catheter. In some cases the obturator has an effective stiffness that is 1.5 lb-in² (e.g., 1.5 lb-in² or less, 1.4 lb-in² or less, 1.3 lb-in² or less, 1.2 lb-in² or less, 1.1 lb-in² or less, 1.0 lb-in² or less, 0.9 lb-in² or less, 0.8 lb-in² or less, 0.7 lb-in² or less, etc.). In some cases the obturator may be highly flexible (e.g., may have a low effective stiffness) over the proximal length, but the distal-most x mm may have a significantly higher stiffness (although a tapered portion configured to extend distally out of the catheter may again be highly compliant/have a low effective stiffness), and may be shape-set in a predefined shape (e.g., straight, bent, curved, etc.). For example, the portion of the obturator between a tapered distal end region (configured to extend distally out of the catheter) and the extending between 0.5 cm and 10 cm, between 0.5 cm and 9 cm, between 0.5 and 8 cm, between 0.5 and 7 cm, between 0.5 and 6 cm, between 0.5 and 5 cm, between 0.5 and 4 cm, etc.) may be shape-set and/or may have an effective stiffness that is greater than a stiffness of the distal end regio of the catheter, such as an effective stiffness of greater than about 1.5 lb-in² (e.g., 1.6 lb-in² or more, 1.7 lb-in² or more, 1.8 lb-in² or more, 1.9 lb-in² or more, 2.0 lb-in² or more, 2.2 lb-in² or more, 2.5 lb-in² or more, 3.0 lb-in² or more, etc.).

As mentioned, any of these apparatuses may include a second input to release pressure from the rigidizing apparatus (e.g., from between the wall(s) of the apparatus). For example, the second input may be configured to release pressure from the wall of the rigidizing catheter by a rate of less than 50 mmHg/sec.

The apparatuses (e.g., systems) described herein, may include the rigidizing catheter, one or more additional catheters (e.g., aspiration catheters), rigidizing pressure source (e.g., insufflator, etc.), and vacuum line components (e.g., vacuum activation valve, clot capture chamber, etc.) and may be configured to remain within the sterile field during a medical procedure using these apparatuses. For example, the rigidizing catheter may be positioned within the body and used for multiple introductions and removal steps, typically without requiring the use of a guidewire, e.g., to reposition the distal end of the rigidizing apparatus (which may be referred to as an overtube in some examples). Aspiration without a guidewire enables larger luminal area (and therefore greater suction), it reduces guidewire risks, and enables for smoother clot transit, because the clot does not need to shear relative to the wire. This may allow the system to be operated within the limited sterile filed during the entire procedure.

Because a rigidized cannula creates a fulcrum at its tip, the behavior of otherwise flexible aspiration catheters or other flexible instruments is altered accordingly. Unlike a standard system that must be navigated and controlled from the access site (e.g. femoral artery), a device that is manipulated within a rigidized cannula has different control dynamics. The navigable portion of a flexible device is shortened, leading to improved local control. In addition, because of the repositioned fulcrum, the tip of the rigidizing cannula can be used as a pivot point. In this case, a tensioning member within the flexible device can be tensioned to cause the flexible device to flex at the fulcrum. Since the flexible device can be moved in and out of the device, the pivot point moves accordingly. A tensioning member within the flexible device can alter the orientation of the tip thereby improving its navigability.

In general, these methods and apparatuses may be used as part of any appropriate medical procedure, in particular vascular procedures, such as, but not limited to, clot removal from the pulmonary vasculature, neurovasculature, coronary vasculature, or peripheral vasculature.

The rigidizing catheters may include an elongate flexible body that is configured to rigidize along all or portion of the length of the body. The elongate flexible body may generally be an elongate tubular body, and may be any appropriate length (e.g., between 0.5 m and 2 m, between 0.7 m and 1.5 m, etc.), and any appropriate outer diameter (e.g., between 4 French and 35 French, between 10 French and 28 French, between 20 French and 30 French, etc.). The elongate flexible body may be coated inside or outside with a lubricious (e.g., hydrophilic) coating or a hydrophobic coating (for example, parylene). The elongate flexible body may include a plurality of layers, including one or more support layers. For example, the plurality of layers may comprise an inner and/or outer wound coil layer. The coil wound layer may support the rigidizing layer(s) and/or may be configured to withstand the application of suction through the lumen.

Any of the rigidizing apparatuses, including the rigidizing catheters and (in some examples) the aspiration catheters, described herein may include rigidizing layers or regions that engage with a compression layer (which may be or may include a bladder, e.g., “bladder layer”) that applies force to the rigidizing layer to rigidize the rigidizing layer or in some cases to de-rigidize (e.g., release from rigidization) the rigidizing layer. In some examples, these rigidizing apparatuses may include a rigidizing layer that could include a braid, knit, woven, chopped segments, randomly distributed or randomly oriented filaments or strands, engagers, links, scales, plates, segments, particles, granules, crossing filaments, or other materials forming the rigidizing layer. For example, the rigidizing layer may comprise multiple strand lengths or strand segments that cross over each other (e.g., as part of a braid, knit, woven, etc.); the compression layer may apply force to drive the crossing strand lengths or strand segments against each other. Although many of the examples shown herein are braids, any of these apparatuses may instead or in addition include a general rigidizing layer comprising crossing strand lengths or strand segments. The examples of rigidizing apparatuses described herein may use positive pressure and/or negative pressure (vacuum) to selectively and controllable rigidize. The examples of rigidizing apparatuses described herein may use pressure (positive pressure) together with negative pressure (vacuum), for example, by ‘pushing’ with positive pressure on one side of a membrane, and ‘pulling’ with vacuum on the other side of a bladder.

In general, the rigidizing catheters described herein may include a lumen, which may be central or may be offset, that is configured to allow passage of a guidewire, a dilator, contrast, a divided lumen (‘double lumen’), and one or more additional devices (e.g., a suction catheter). In some examples the rigidizing catheters described herein include a lumen that includes a lubricious internal coating or layer, and/or is formed of a lubricous material, such as a hydrophilic material. The lumen of the rigidizing catheters may be any appropriate size (e.g., inner diameter). For example, the rigidizing catheters may have an inner diameter that is between about 4 French and 35 French, between about 10 French and 20 French, between about 12 French and 18 French, etc. The inner diameter may be substantially uniform along the length of the lumen. In some examples the inner diameter may include one or more narrowing (tapered) regions along the length. For example in some examples the distal end region of the lumen may be conical, cone-shaped, or tapered. The geometric change may be tapered towards a larger diameter, or towards a smaller diameter. This geometric change may be a stable geometry, or it may be a compressible or collapsible geometry, for example, a nitinol-based conical structure that has an overlaid membrane (e.g., skin).

As mentioned, any of the rigidizing catheters described herein may be configured to apply suction through the lumen of the rigidizing catheter. In some cases, the rigidizing catheter may include a mating attachment connector proximal to the hemostasis valve region and configured to lockingly engage with a mating attachment of a suction line. This mating attachment connector may be a universal connector that is similar, or identical to, the mating attachment connector of the aspiration catheter. Thus, the same suction line may be attached (swapped between) the aspiration catheter and the rigidizing catheter. Alternatively in some examples, the suction may be applied to both the rigidizing catheter and an aspiration catheter within the lumen of the rigidizing catheter (e.g., around the aspiration catheter).

Any of the rigidizing catheters described herein may include an atraumatic tip at the distal end of the rigidizing aspiration sheath catheter. For example, the distal end (tip) region) may be formed of a relatively soft (e.g., low durometer) material and may be rounded.

As mentioned, the rigidizing catheters described herein may be configured to rigidize by the application of pressure; for example, the application of positive or negative (vacuum) pressure. In some examples the elongate flexible body of the rigidizing catheter may be configured to transition from the flexible state to the rigid state upon application of positive pressure. In some examples the elongate flexible body is configured to transition from the flexible state to the rigid state upon application of negative pressure. In some examples the elongate flexible body is configured to transition from the flexible state to the rigid state upon application of negative pressure to one side of a bladder simultaneous with the application of positive pressure to the other side of a bladder.

Also described herein are systems including any of these rigidizing catheters. Each of the components of these systems may be used separately or in combination. These systems may include multiple versions of any of these components. For example a cardiovascular aspiration system (e.g., a system for aspiration of clot material) may include: any of the rigidizing catheters described herein, any of the aspiration catheters described herein, and any of the suction lines (including all or some of the suction line components described herein).

The rigidizing catheter systems described herein may include: a rigidizing catheter that is configured to transition from a flexible state to a rigid state upon application of pressure within a wall of the rigidizing catheter; and a pressurizing device that is configured to supply a flow of fluid for applying the pressure within the rigidizing catheter, wherein the pressurizing device includes a control that is configured to control a pressure change within the rigidizing catheter by a rate ranging from about 10 to about 200 mmHg/sec (or any subrange therebetween, e.g., between 10 and 150 mmHg/sec, between 10 and 100 mmHg/sec, between 10 and 90 mmHg/sec, etc.), thereby allowing gradual transition between the flexible and rigid states of the rigidizing catheter.

In some examples the aspiration catheter and/or the rigidizing catheter is/are steerable by one or more actuating steering members. The actuating steering members may be any appropriate steering member, including mechanical steering members (e.g., one or more tendons, cables, wires, etc., actuators, etc.), pneumatic steering members, magnetic steering members, thermal steering members (e.g., using a shape memory alloy or shape memory polymers, etc.). Although the actuating steering members examples described herein primarily include one or more cables, any appropriate actuating steering member may be used in any of these apparatuses and methods.

The rigidizing catheters described herein may be used as sheaths or overtubes for one or more other catheters. For example, as discussed, the rigidizing catheter may serve as a rigidizing aspiration sheath catheter for an aspiration catheter, where the aspiration catheter is able to advance through the rigidizing aspiration sheath catheter that is positioned within the subject's body (e.g., within the vasculature).

Methods may include removing a clot material from a vasculature. For example, a method for removing clot material may include: advancing a rigidizing aspiration sheath catheter in a vessel while the rigidizing aspiration sheath catheter is in a flexible state; transitioning the rigidizing aspiration sheath catheter from the flexible state to a more rigid state by applying pressure within the rigidizing aspiration sheath catheter, wherein the pressure is maintained a first pressure; steering a distal end region of the rigidizing aspiration sheath catheter by changing the pressure from the first pressure to a second pressure, wherein the pressure is gradually changed until the distal end region of the rigidizing aspiration sheath catheter is moved to a desired position near the clot material; rigidizing the rigidizing aspiration sheath catheter once the distal end region has moved to the desired position by re-applying pressure within the rigidizing aspiration sheath catheter; and removing the clot material from the vessel by aspirating through the rigidizing aspiration sheath catheter.

Any of these methods may include visualizing the rigidizing catheter while being steered or otherwise positioned within a subject's body. For example, the rigidizing catheter, and especially a distal end region of the rigidizing catheter, may be visualized using fluoroscopic (X-ray) imaging, ultrasound (e.g., Doppler) imaging, computed tomography angiography (CTA) imaging, magnetic resonance angiography (MRA) imaging and/or optical coherence tomography (OCT) imaging.

As mentioned, these apparatuses may be used with or without a guidewire, either for the entire procedure or for the portion of the procedure following initially placing the rigidizing catheter (e.g., rigidizing overtube). For examples, advancing the rigidizing catheter may include advancing the rigidizing catheter without the use of a guidewire. In some examples advancing the rigidizing catheter comprises advancing the aspiration catheter distally relative to the rigidizing catheter in the rigid state and steering a distal end of the aspiration catheter. The method may further include advancing the rigidizing catheter in the flexible state over the aspiration catheter and rigidizing the rigidizing catheter. In some examples advancing the rigidizing catheter comprises advancing the rigidizing catheter with an obturator within a lumen of the rigidizing catheter.

The methods described herein may be methods for treatment of any cardiovascular disorder, including, but not limited to removing clot material from an artery or a vein, including but not limited to the neurovasculature, the coronary vasculature, the peripheral vasculature, or the pulmonary vasculature, including the pulmonary artery. For example, advancing the rigidizing catheter to the treatment location may include advancing a distal end of the rigidizing catheter to a pulmonary artery of a patient.

Although in general, these methods and apparatuses describe the use of a rigidizing catheter (e.g., rigidizing overtube) and one or more non-rigidizing aspiration catheters that may extend from the rigidizing catheter, any of these methods and apparatuses may include a rigidizing (e.g., pressure rigidizing) aspiration catheter. Thus, in some cases both the rigidizing catheter and the aspiration catheter may be rigidizing by the application of pressure (positive and/or negative pressure).

In general, these methods and apparatuses may hold the rigidizing overtube in a rigid state while applying suction (aspiration) through one or both of the rigidizing overtube and/or an aspiration catheter. This may be particularly advantageous as it may prevent movement of the rigidizing catheter (e.g., rigidizing overtube) within the lumen of the vessel, even when the distal tip of the rigidizing catheter is near the vessel wall, and even at relatively high suction (e.g., flow rate). This may prevent latching of the rigidizing catheter to the wall. Rigidizing the rigidizing catheter may also prevent inadvertent movement of the aspiration catheter that may otherwise occur within the vessel when aspiration is applied.

A mentioned above, the methods and apparatuses described herein may control the different stiffnesses for navigation and for treatment, and in particular, may be configured to have effective stiffnesses within a predefined range that is effective; outside of the indicated ranges of effective stiffnesses these catheters may not work well, or at all. For example, a dynamically rigidizing apparatus (including but not limited to dynamically rigidizing catheters and other components) as described herein can have a substantially lower effective stiffness (in lb-in²) over its entire length in a flexible configuration that may result in less of a load on localized anatomical features during navigation, while the same rigidizing catheter(s) may convert, without substantially foreshortening or shifting position, into a rigid configuration that is sufficiently rigid (e.g. has an effective stiffness along its length) to hold the catheter in a shape that avoids damage to the anatomy through which it has been navigated, even when passing a second catheter or tool that may tend to push against the rigidizing catheter.

The inner aspiration catheters described herein may be configured to operate with the outer rigidizing catheter. Specifically, the inner aspiration catheters may include a region of extremely high flexibility at the distal end of the catheter (e.g., from the distal tip, or a region just proximal to the distal tip, which may be pre-bent, to a proximal region) may be configured to have a very low effective stiffness (e.g., 1.5 lb-in² or less). Unlike contemporary aspiration catheters, particularly commercially available catheters, a long length of the catheter may have the same, high flexibility (low effective stiffness), allowing the catheter to extend distally out of the rigid outer rigidizing catheter to capture clot material. Because these aspiration catheters may be supported by the rigidizing outer catheter, the length of the highly flexible region may be significantly longer (e.g., relative to the length of the full catheter), such as 10 cm or greater (15 cm or greater, 20 cm or greater, 25 cm or greater, 30 cm or greater, 35 cm or greater, 40 cm or greater, 50 cm or greater 55 cm or greater, 60 cm or greater, 65 cm or greater, 70 cm or greater, 75 cm or greater, 80 cm or greater, 85 cm or greater, 90 cm or greater, 95 cm or greater, etc.), which is unusual in contrast to contemporary aspiration catheters that must have a significantly higher effective stiffness in order to avoid buckling, bending or unguided tip movement, particularly when advancing the catheter.

The inner aspiration catheters described herein may include a significantly more rigid proximal end region (e.g., proximal 60%, proximal 55%, proximal 50%, proximal 45%, proximal 40%, proximal 35%, proximal 30%, proximal 25%, proximal 20%, etc.) which may enhance or simplify loading of the inner aspiration catheter into the outer rigidizing catheter. Further, it may be beneficial to abruptly transition (e.g., over less than 10 cc, less than 8 cm, less than 7 cm, less than 5 cm, less than 4 cm, less than 3 cm, less than 2 cm, less than 1 cm, etc.) from the highly flexible distal end region (distal region) to the significantly more rigid proximal end region (proximal region). The difference between the effective stiffness of the highly flexible distal region and the more rigid proximal region may be between 1.25× and 100× different (e.g., between about 1.5× and 100×, between about 1.75× and 50×, between about 2× and 50×, between about 2.25× and 40×, or any subregion therebetween). This abrupt transition may enhance pushability and may be significantly easier and less expensive to fabricate. Further, the abrupt transition is not useful (and indeed may result in kinking and/or harm to the patient) without the use of the outer rigidizing catheter as described herein. For example, the highly flexible proximal region may be immediately adjacent to the distal, less flexible region so that effective bending stiffness transitions abruptly by more than 0.5 lb-inch².

An apparatus (e.g., system) for removing clot may include: an outer rigidizing catheter having a flexible state and a rigid state; and an inner aspiration catheter configured to extend through and distally out of the outer rigidizing catheter, wherein the inner aspiration catheter comprise a highly flexible proximal region that extends at least x % (e.g., at least 35%, at least 40%, at least 45%, at least 50%, at least 55%, at least 60%, at least 65%, at least 70%, at least 75%, at least 80%, at least 85, etc. including any sub-range therein) of a length of the inner aspiration catheter and has an effective bending stiffness of about 1.5 lb-inch² or less (e.g., 1.5 lb-in² or less, 1.4 lb-in² or less, 1.3 lb-in² or less, 1.2 lb-in² or less, 1.1 lb-in² or less, 1.0 lb-in² or less, 0.9 lb-in² or less, 0.8 lb-in² or less, 0.7 lb-in² or less, etc.), and wherein the inner aspiration catheter comprises a distal, less flexible region having an effective bending stiffness that is greater than about 2 lb-inch². In some examples the distal, less flexible region of the inner aspiration catheter may have an effective bending stiffness that is greater than about 3 lb-inch², so that the effective bending stiffness transitions abruptly by more than 1.5 lb-inch².

The outer rigidizing catheter may have a length that is about 1 meter or longer (e.g., 1.1 m or longer, 1.2 m or longer, 1.3 m or longer, 1.4 m or longer, 1.5 m or longer, etc.). In some cases the outer rigidizing catheter is configured to extend between a femoral access point to a pulmonary artery. The inner aspiration catheter is typically longer than the outer rigidizing catheter, and may be up to 10% longer or more (e.g., 15% longer or more, 20% longer or more, 25% longer or more, 30% longer or more 35% longer or more, between about 5%-50% longer, 10%-30% longer, etc.). For example, the inner aspiration catheter is configured to extend distally out of the outer rigidizing catheter by more than about 5 cm.

The maximum effective bending stiffness of the outer rigidizing catheter in the flexible configuration may be about 3 lb-inch² or less (e.g., 2.5 lb-inch² or less, 2.25 lb-inch² or less, 2.15 lb-inch² or less, 2.0 lb-inch² or less, 1.85 lb-inch² or less, 1.75 lb-inch² or less, 1.65 lb-inch² or less, 1.5 lb-inch² or less, 1.4 lb-inch² or less, 1.3 lb-inch² or less, 1.2 lb-inch² or less, 1.1 lb-inch² or less, 1.0 lb-inch² or less, etc.) and the minimum effective bending stiffness in the rigid configuration may be about 25 lb-inch² or greater (e.g., 27.5 lb-inch² or more, 30 lb-inch² or more, 32.5 lb-inch² or more, 35 lb-inch² or more, 37.5 lb-inch² or more, 40 lb-inch² or more, 45 lb-inch² or more, 50 lb-inch² or more, etc.).

In general, the outer rigidizing catheter and the inner aspiration catheter may be sized and configured for use within the peripheral and/or neurovasculature. Thus, the outer diameter of the outer rigidizing catheter and the inner aspiration catheter may be, e.g., between 3 F (or smaller) and 36 F (or greater). The outer rigidizing catheter is typically larger (by between 2-5 F) than the inner aspiration catheter, and may have an inner diameter that is the same size or slightly larger than the outer diameter of the inner aspiration catheter. For example, the outer rigidizing catheter may be between about 15 F and 28 F in some examples; the inner aspiration catheter may be between about 8 F and 22 F.

In general, the inner aspiration catheter may comprise a distal tip region comprising a bent or curved or angled region. The apparatus (e.g., system) may include an obturator for the inner aspiration catheter that can unbend/un-curve the distal end for insertion and position of the inner aspiration catheter. As mentioned, any of these apparatuses may also include one or more obturators for the outer rigidizing catheter. The stiffness (e.g., the effective stiffness) of the obturators may be matched to the effective stiffness of the catheter; for example, the effective stiffness of the obturator for the outer rigidizing catheter may be configured to be equal to or less than the effective stiffness of the outer rigidizing catheter in the non-rigid (e.g., flexible) state. For example.

In any of these apparatuses, the outer rigidizing catheter may comprise a rigidizing layer and a bladder layer, further wherein the outer rigidizing catheter is configured to convert from the flexible state to the rigid state by the application of positive and/or negative pressure to drive the bladder layer against the rigidizing layer to limit shearing of the rigidizing layer.

For example, described herein are systems for removing clot that include: an outer rigidizing catheter having a flexible state and a rigid state, wherein the outer rigidizing catheter has a length that is about 1 meter or longer, further wherein the maximum effective bending stiffness of the outer rigidizing catheter in the flexible configuration is about 3 lb-inch² or less and the minimum effective bending stiffness in the rigid configuration is about 25 lb-inch² or greater; and an inner aspiration catheter that is configured to extend distally out of the outer rigidizing catheter, wherein a distal region of the inner aspiration catheter that extends at least 40% of a length of the inner aspiration catheter is highly flexible and has a bending stiffness of about 1.5 lb-inch² or less. The outer rigidizing catheter may be configured to extend between a femoral access point to a pulmonary artery. The maximum effective bending stiffness of the outer rigidizing catheter in the flexible configuration may be about 2.1 lb-inch² or less and the minimum effective bending stiffness in the rigid configuration is about 30 lb-inch² or greater. The distal end region is bent or curved. The distal end region may have a maximum effective bending stiffness that is greater than 1.5 lb-inch². A region of the inner aspiration catheter extending from a proximal end to the distal region may be less flexible than the distal region and has a minimum effective bending stiffness of greater than 2 lb-inch² or more. The inner aspiration catheter may be between about 15 F and 28 F. The inner aspiration catheter may be configured to extend distally out of the outer rigidizing catheter by more than about 5 cm.

Also described herein are methods of using any of these apparatuses. In some cases these methods may include methods of removing clot material. For example, a method may include: positioning an outer rigidizing catheter within a patient's vasculature in a flexible configuration in which the outer rigidizing catheter has a maximum effective bending stiffness of less than about 3 lb-inch²; applying positive and/or negative pressure to rigidize the outer rigidizing catheter to a minimum effective bending stiffness of about 25 lb-inch² or greater; maintaining the outer rigidizing catheter in the rigid state while inserting a flexible inner aspiration catheter distally through and out of the outer rigidizing catheter to prevent buckling of the flexible inner aspiration catheter; and removing clot material from a region distal to a distal end of the outer rigidizing catheter.

Positioning the outer rigidizing catheter may comprise sliding the outer rigidizing catheter over a guidewire. In some cases positioning the outer rigidizing catheter comprises inserting an obturator into the outer rigidizing catheter, wherein the obturator has a maximum effective bending stiffness of less than about 3 lb-inch². Positioning the outer rigidizing catheter within the patient's vasculature may comprise positioning a distal end region of the outer rigidizing catheter distal to a bifurcation in the pulmonary artery. In some cases, removing clot material comprises rotating the flexible inner aspiration catheter within the outer rigidizing catheter to orient a bent or curved distal end of the flexible inner aspiration catheter towards a clot material. For example, inserting the flexible inner aspiration catheter distally may comprise inserting the flexible inner aspiration catheter further than 5 cm distal to a distal end of the outer rigidizing catheter. The flexible inner aspiration catheter may have a maximum effective bending stiffness from a distal tip region to a proximal region that is more than 40% of a length of the flexible inner aspiration catheter that is 1.5 lb-inch² or less.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A shows one example of a system including a rigidizing catheter apparatus as described herein.

FIGS. 1B-1G show examples of a system and components of these systems as described herein.

FIGS. 2A-2B show an example of a portion of a vacuum rigidizing apparatus as described herein. FIG. 2A shows a section through the exemplary vacuum rigidizing member of the apparatus. FIG. 2B shows an enlarged view of a portion of the section, illustrating the arrangement of layers in the un-rigidized configuration.

FIGS. 3A-3D show an example of a portion of a vacuum rigidizing apparatus having multiple rigidizing layers as described herein. FIG. 3A shows a perspective view of the vacuum rigidizing member with the outer layer removed (showing the outermost rigidizing layer). FIG. 3B is an enlarged view of a portion of FIG. 3A. FIG. 3C shows a longitudinal section though the vacuum rigidizing member of FIG. 3A. FIG. 3D is a cross-section through the rigidizing member of FIG. 3A.

FIGS. 4A-4B show an example of a pressure rigidizing device.

FIG. 5 shows an example of a rigidizing device with a distal end section.

FIG. 6 shows an example of a rigidizing device with a distal end section having a plurality of actively controlled linkages.

FIG. 7 schematically illustrates an example of a section through a cross-section on one example of a proximal end of a rigidizing catheter (also referred to herein as simply a rigidizing catheter).

FIGS. 8A and 8B show a traditional catheter and a rigidizing catheter, respectively, navigating to the pulmonary artery.

FIGS. 9A-9D illustrate one example of a method of steering a distal end region of a rigidizing catheter (e.g., an outer rigidizing catheter) by controlling the rigidity of the catheter. FIG. 9A shows the outer rigidizing catheter in a rigid configuration. FIG. 9B show the apparatus of FIG. 9A while the is gradually released, so that the tip of the outer rigidizing catheter moves slowly towards a second position. FIGS. 9C and 9D illustrate re-rigidizing the outer rigidizing catheter and further releasing the pressure (rigidizing the catheter) to further move the distal end/tip.

FIGS. 10A and 10B show an example of steering a distal end region of a rigidizing catheter within a vasculature by changing the rigidizing pressure applied to the rigidizing catheter.

FIG. 11A illustrates a method of determining a deflection force and therefore effective stiffness of a catheter as described herein.

FIG. 11B is a table showing experimental and calculated values of deflection force and effective stiffness for various examples of outer rigidizing catheters, inner aspiration catheters, and commercial catheters (including commercial aspiration catheters).

FIG. 11C schematically illustrates an example of an apparatus including an outer rigidizing catheter and an inner aspiration catheter having one beneficial arrangement of effective stiffnesses along the lengths of the outer rigidizing catheter and inner aspiration catheter.

### DETAILED DESCRIPTION

Described herein are methods and apparatuses (e.g., devices, systems, assemblies, etc.) that may be used as part of a vascular procedure, including for clot removal. These methods may include an outer rigidizing catheter, which may be controllably transitioned between a rigid configuration and highly flexible configuration. These methods and apparatuses may also include one or more inner aspiration catheters and/or one or more obturators, as well as one or more pressure controllers, fluid lines (e.g., tubing), flush lines, suction/aspiration sources, clot capture sources, etc. In any of these methods and apparatuses the outer rigidizing catheter may be configured to be steered by controlling the release (e.g., gradual release) of rigidizing pressure (positive and/or negative pressure). Also described herein are apparatuses and methods of making and using them, that control the effective stiffnesses of the outer rigidizing catheter and/or the inner aspiration catheter (and/or the obturator(s) for either or both of these) to enhance safety and efficacy of these apparatuses.

The rigidizing catheters are configured to transition between flexible and rigid states through the application of positive and/or negative pressure applied within walls of the rigidizing catheters. In some examples a distal end region (e.g., tip) of the rigidizing catheters may be precisely steered toward a target region of subject's body by controllably changing the rigidity of the outer rigidizing catheter, e.g., by controllably changing the pressure within the walls of the rigidizing catheter or catheters. Controlled changing of the pressure can provide a controlled change in shape of the rigidizing catheter as the rigidizing catheter relaxes from the rigid state to a flexible state. In some cases the outer rigidizing catheter may be biased to have a relaxed configuration (e.g., weakly biased) and/or may be used with a tool, such as, but not limited to, an obturator so that relaxing the outer rigidizing catheter from a rigid to a flexible configuration by gradually releasing pressure maintaining the outer rigidizing catheter in a rigid state, may controllably move the distal end region of the outer rigidizing catheter as shown and described herein.

These methods and apparatuses may be well suited for use in systems configured for the removal of clot material (e.g., for clot capture) by aspiration. The rigidizing catheters can provide a platform for the aspiration system. Using a rigidizing catheter to navigate to locations within the vasculature, for example deep within the vasculature, can advantageously allow for creation of a stable pathway to the treatment site through which aspiration can be performed and allow precise steering of the rigidizing catheter, especially of a distal end region of the rigidizing catheter. These advantages simplify procedures, greatly improve outcomes, and increase patient safety.

In general, these methods and apparatuses may include a rigidizing (e.g., dynamically rigidizing) catheter that may include an elongate flexible body having a plurality of layers, including one or more rigidizing layer and a compression layer (e.g., a bladder layer) configured to transition the rigidizing layer between a flexible state and a rigid state. The rigidizing catheter may include a lumen extending through the elongate flexible body and an integrated hemostasis valve. The integrated hemostasis valve may be part of a hemostasis valve region at a proximal end of the elongate flexible body.

The dynamically rigidizing catheters described herein may be controllable switched between a highly flexible state and one or more (or a continuously increasing) stiff or rigid state(s). In some examples the rigidizing catheter may be configured so that the flexible state is the resting state (e.g., without a pressure differential being applied). In general, the flexible state may be a low resting energy state. That is, in its flexible state the catheter may exert very low forces on the anatomy. In the rigid state(s) the outer rigidizing catheter may exert low forces on the anatomy, and when the catheter is released (either intentionally or accidentally) it may still have a low potential energy. Thus, the catheter may avoid or prevent movement recoil (e.g., “kick”, which may occur when transitioning to applying suction), and may stay in the same position. This may be particularly beneficial as compared to non-rigidizing devices that may have higher energy states (sometimes much higher), and that apply a recoil force that can injure the patient when released (either intentionally or accidentally).

The rigidizing catheters and systems described herein may include a pressurizing device that is configured to supply and/or withdraw fluid (e.g., gas, liquid) to and/or from the walls of the rigidizing catheter. The pressurizing device may be configured to provide positive and/or negative (vacuum) to the rigidizing catheter. The pressurizing device may be configured to provide a controlled flow of fluid (e.g., gas, liquid) to and/or from the rigidizing device. In some cases, the pressurizing device may be in the form of an insufflator. In some examples, the pressurizing device may be configured to allow the user to gradually increase and/or decrease the pressure. For example, it may be desirable to change the pressure within the rigidizing device gradually to allow the rigidizing device to relax or rigidize to a desired shape, as described herein. In some examples, the pressurizing device may be configured to change the pressure at a rate ranging from about 5 mmHg/sec to about 200 mmHg/sec (or any subrange therebetween, e.g., between 10 and 150 mmHg/sec, between 10 and 100 mmHg/sec, between 10 and 90 mmHg/sec, etc.)

The pressure source device may include one or more pressure sensors to detect (e.g., monitor) the pressure being applied and/or withdrawn from the rigidizing device. In cases where a water solution is used (e.g., saline), the pressure sensor may be configured to measure hydrostatic pressure. The pressurizing device may include manual and/or digital controls.

These methods and apparatuses may also include an aspiration catheter that may be inserted into the rigidizing catheter, which may also include a hemostasis valve region. The aspiration catheter may be steerable by an actuating steering member (e.g., pull wire, tendon, etc.) integrated into the catheter and/or held within a lumen (e.g., working channel, aspiration channel, etc.) of the catheter. In some examples the aspiration catheter may be rigidizing as well.

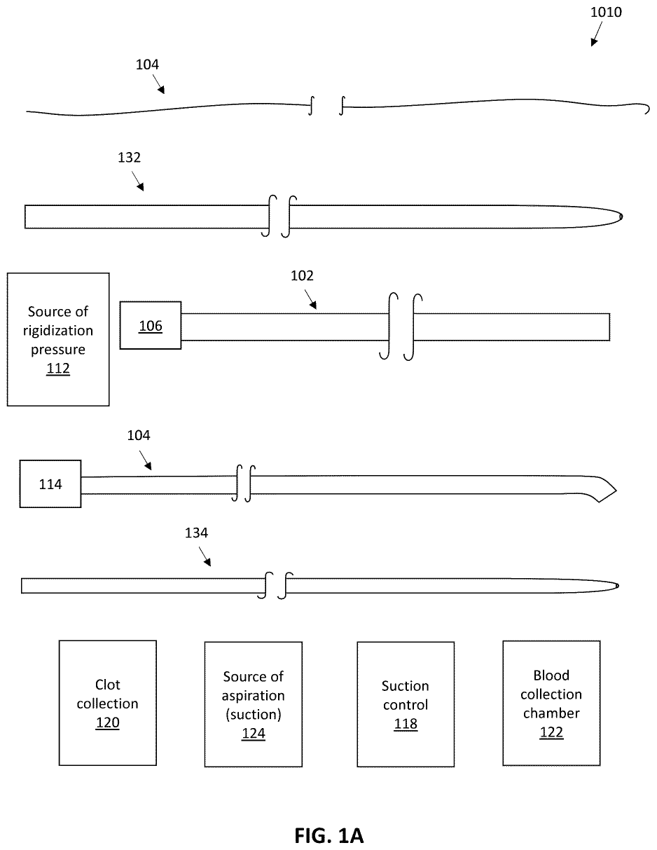

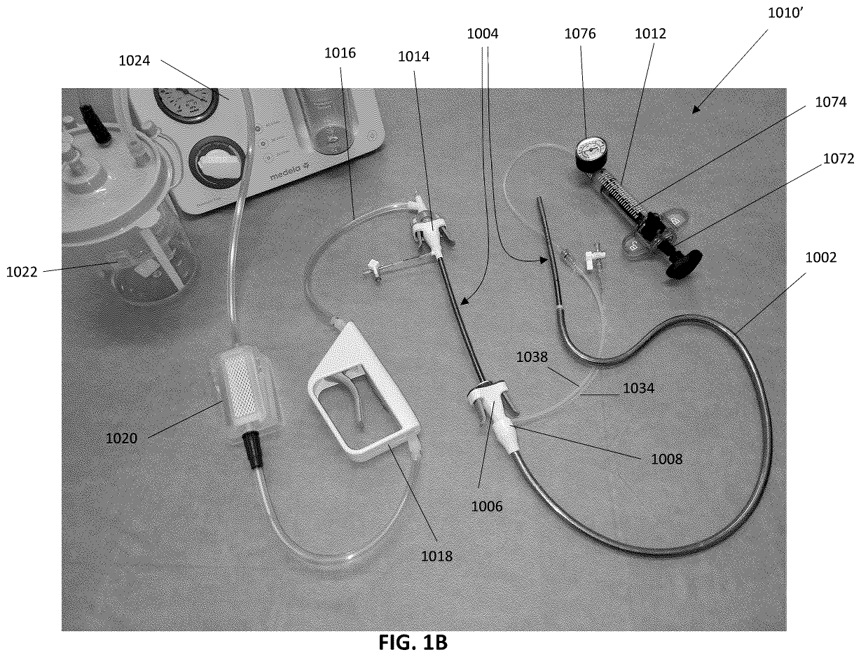

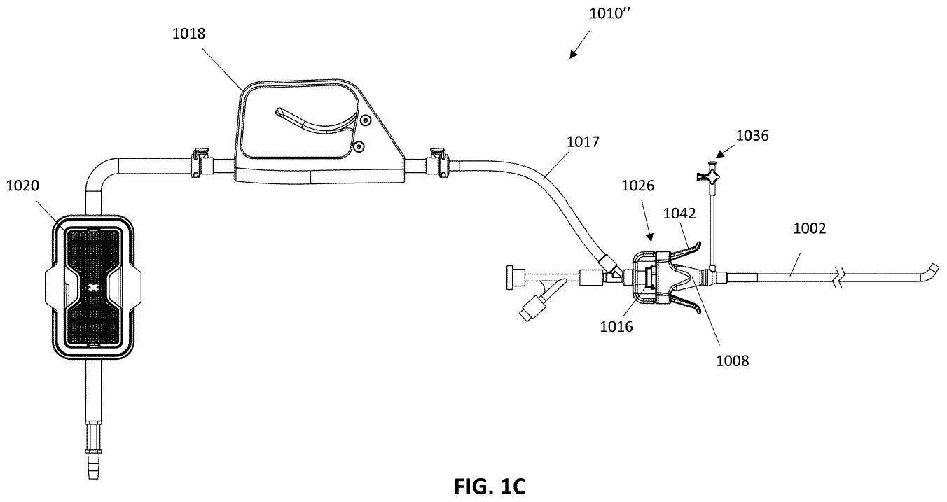

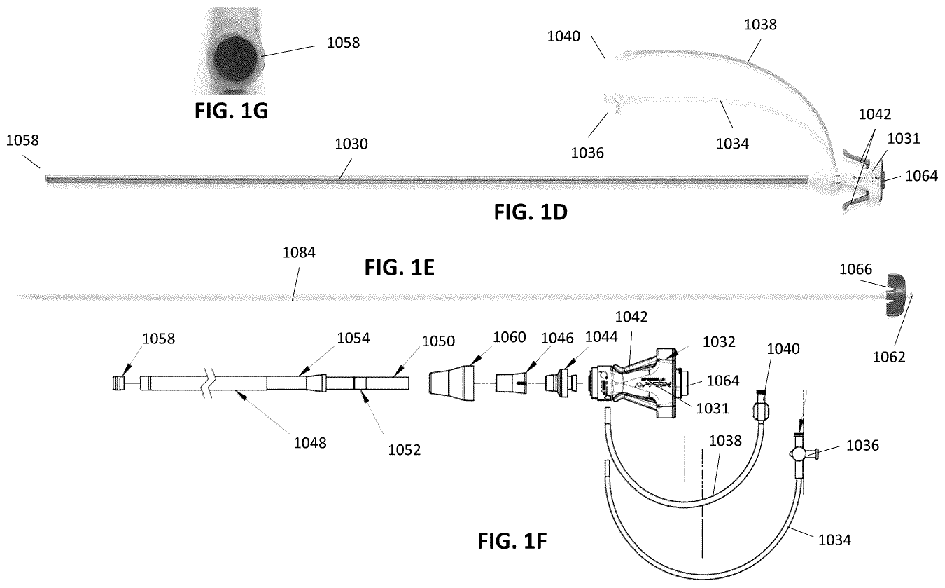

The method and apparatuses described herein may include a vacuum line with one or more of: a hand-triggered vacuum activation valve and/or a clot capture chamber for visualizing and removing clot material aspirated by the system. FIG. 1A schematically illustrates one example of an apparatus (e.g., system) including at least a rigidizing catheter 102 including a proximal region configured as a hemostasis valve region 106. The rigidizing catheter is generally configured to be changed between a flexible state (flexible configuration) and a less flexible, e.g., rigid, state (rigid configuration). Any appropriate structure for rigidizing may be used, including in particular a layered structure that is rigidized by the application of positive and/or negative pressure. For example, these apparatuses may be configured as a rigidizing catheter configured to couple to a source of positive and/or negative pressure 112, e.g., through a port or inlet on the proximal end (which may be part of the hemostasis valve region or separate from it) to control the rigidity of the rigidizing catheter. In some examples the rigidizing catheter apparatus has an elongate body comprising a lumen extending therethrough. The elongate body may include layers, such as a rigidizing layer and a bladder layer that are configured to transition the elongate body between a flexible state and a rigid state by the application of pressure. As described in greater detail below, the bladder layer (e.g., “bladder”) may be driven against (or allowed to move away from) the rigidizing layer to control the flexibility/stiffness of the elongate body. The rigidizing layer may comprise a plurality of overlapping filament lengths that are free to slide over each other in the more flexible state(s) of the rigidizing catheter, but are prevented or limited from sliding over each other when the bladder layer is driven against the rigidizing layer, rigidizing the elongate body. Positive pressure may be applied to the bladder layer to drive the bladder layer against the rigidizing layer and/or negative pressure may be applied to pull the bladder layer against the rigidizing layer to rigidize the device. In some cases a combination of positive and/or negative pressure may be applied.

The rigidizing catheter may be used with one or more obturators 132. In FIG. 1A the obturator may be inserted into the rigidizing catheter over a guidewire 104 (which may be included with the apparatus 1010 or may be separately provided). This may allow the rigidizing catheter to be guided over a guidewire positioned in a body vessel. The obturator may be steerable or not. In general, the obturator may be flexible so that the combined obturator and rigidizing catheter (in the flexible configuration) may readily track over a guidewire. The obturator may be longer than the rigidizing catheter (e.g., by more than 1 cm (e.g., between 1 cm-20 cm, between 1 cm-30 cm, 1 cm-40 cm, etc. or any number therebetween) to allow tracking while avoiding “fishmouthing” over the rigidizing catheter distal end opening. The obturator may have an atraumatic tip. The obturator may have regions of different material properties (e.g., stiffnesses), such as described in PCTUS2022082141, filed Dec. 21, 2022, and herein incorporated by reference in its entirety.

The apparatus 1010 (e.g., system) in FIG. 1A may also include one or more aspiration catheters 104 that may also include a proximal hemostasis valve region. The aspiration catheter may also be used with an obturator 134 that may be inserted through the aspiration catheter and inserted through the rigidizing catheter, e.g., in the rigid configuration. In some cases it may be beneficial for the distal tip region of the aspiration catheter to be directional (e.g., bent, curved, etc.) in a fairly rigid bend, to allow for directional aspiration when extended from the rigid rigidizing catheter. The aspiration catheter may generally be configured to have a relatively high flexibility with a high torquability. The high torquability may allow the apparatus to be steered (directed) within the rigidizing catheter when extended distally of the distal end of the rigidizing catheter, e.g., in the rigid configuration.

Other system components may include tubing (suction line) connecting the rigidizing catheter and/or aspiration catheter to a source of aspiration 124. The rigidizing catheter and/or aspiration catheter may be connected via a sealing connection to the suction line, which may be connected in-line to a clot collection chamber 120, and/or a suction (e.g., vacuum) activation valve, which may be activated to apply suction to the rigidizing catheter and/or aspiration catheter. The apparatus may also include a blood collection chamber 122 before the source of aspiration 124 (e.g., suction pump). These components may be arranged between the rigidizing catheter and/or aspiration catheter and the source of aspiration in any appropriate order.

For example, FIG. 1B illustrates an example of an apparatus (e.g., a system) for clot aspiration including these components. In FIG. 1B the system 1010′ includes a rigidizing catheter 1002 that is shown coupled to an insufflator 1012 to control transitioning between a rigid state and a flexible state. The aspiration system 1010′ also includes an aspiration catheter 1004 shown positioned within and extending out of the rigidizing catheter 1002. The dynamically rigidizing catheter 1002 includes a hemostatic valve region 1006 (which may be integrated into the rigidizing catheter or may be an attached hemostatic valve). The hemostatic valve region includes a connection 1008 to a pressure source (e.g., insufflator 1012). The aspiration catheter 1004 extends proximally from the hemostatic valve region to an aspiration catheter handle 1014, which may be part of the aspiration catheter 1004 or attached thereto. Through the aspiration catheter handle, the aspiration catheter comprises a connection to a tube or other elongate element 1016 that connects to a vacuum activation valve 1018. The tube 1016 extends proximally to a clot capture chamber 1020 via the activation valve 1018. A vacuum pump 1024 is connected at a proximal portion of the blood collection container 1022.

The insufflator 1012 can be configured to provide a gradual change in the pressure applied to the rigidizing catheter 1002 for rigidizing and de-rigidizing the rigidizing catheter 1002. In the example shown, the insufflator 1012 is a syringe-type insufflator that includes a plunger 1072 and a barrel 1047 configured to supply positive pressure (e.g., by pushing the plunger 1072) or negative pressure (e.g., by pulling the plunger 1072) to the rigidizing catheter 1002 via the tube 1038. In some cases, the barrel 1047 includes graduation lines so that a user can determine the volume of fluid (e.g., gas, liquid) that has been injected into or out of the rigidizing catheter 1002. A pressure gauge 1076 may be configured to measure the applied pressure (e.g., positive or negative). If water or saline solution is used, the pressure gauge 1076 may be configured to measure hydrostatic pressure.

Other types of insufflators other than the syringe-type insufflator 1012 may be used. The insufflator may include a mechanism to provide refined controlled release of pressure from the rigidizing catheter. For example, the mechanism may include a corkscrew-type of release valve that provides a more gradual release of pressure, for example by turning a control knob. An insufflator pressure control may be configured to be used with one hand of the user. FIG. 1C shows another example of a portion of a system 1010″ as described herein, including an aspiration catheter 1002 with a hemostatic valve 1026 (which may be integrated or connect to the catheter) and a flush port 1036. The aspiration catheter is shown lockingly coupled to a mating attachment 1016 at a distal end of a suction line (e.g., vacuum line). The mating attachment is configured to couple to a mating attachment connector on the proximal end of the aspiration catheter 1002 for making a quick connection to the suction line 1017. A hand-triggered vacuum activation valve 1018 is shown connected in-line with the vacuum line and may be easily used to turn on/off suction through the apparatus. The vacuum line is also connected to a clot capture chamber 1020, described in greater detail below.

FIGS. 1D-1G show embodiments of components of an aspiration system (e.g., like those shown in FIG. 1B). Referring now to FIGS. 1D and 1F, the system may include a rigidizing catheter 1030 including a hemostatic seal region 1032 at a proximal end. The hemostatic seal region 1032 comprises a body 1031. The seal in this example also includes a tube or elongate element 1034 connecting to a flush port 1036. In some embodiments, the flush port can comprise a luer type adapter. The hemostatic seal region 1032 also includes a tube or elongate element 1038 with a connector 1040 at its proximal end for connection to a pressure source (e.g., an insufflator). The connector may be a luer type connector.

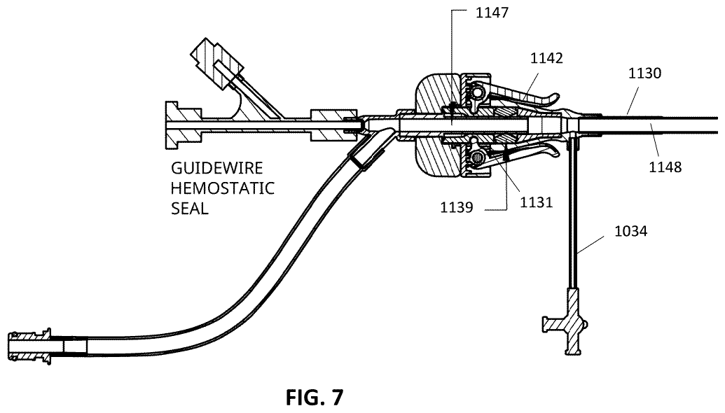

The hemostatic seal region in this example includes a pair of actuators 1042 (shown as levers). Depressing actuators 1042 (e.g., levers, buttons, etc.) can allow for release of a device (e.g., aspiration catheter) positioned within the rigidizing catheter 1030. When the actuators are in their unbiased state, extending outwardly from the body of the seal 1032, the seal valve is closed (shown in FIG. 7D, below).

In FIG. 1F a bladder adapter 1044 for connecting the bladder of the rigidizing catheter 1030 to the hemostatic seal region 1032 is located a distal end of the seal region 1032. This connection allows the seal region 1032 to maintain pressurization (e.g., insufflation) of the rigidizing catheter 1030. Distal to the bladder adapter 1044 is an adapter 1046 for connection to an outer layer 1048 of the rigidizing catheter 1030. A shroud 1060 is located distal to the outer layer adapter 1046, for covering the adapters 1044, 1046 and from which the rigidizing catheter 1030 distally extends.

FIG. 1F further shows an example of the layers of the rigidizing catheter 1030, including the inner layer 1050, the bladder layer 1052, the rigidizing layer 1054 (e.g., in some examples the rigidizing layer comprises a plurality of filaments that cross over each other, such as, but not limited to, a braid layer, knit layer, woven layer, etc.), and the outer layer 1048. At the distal end of the rigidizing catheter 1030 is a distal tip 1058.

FIG. 1G shows an end view of the rigidizing catheter 1030 shown in FIGS. 1D-1F, in which the rigidizing catheter includes an atraumatic, distal tip.

In some embodiments, the rigidizing catheter inner lumen comprises a hydrophilic coating. This coating can help facilitate insertion of an obturator and other devices and accessories.

In some embodiments, an outer surface of the rigidizing catheter comprises a hydrophobic coating. This type of coating can help facilitate smooth motion through an introducer sheath.

The rigidizing catheter may have an inner lumen diameter of about 0.03-0.6 in. In some embodiments, the rigidizing catheter inner lumen diameter is about 0.16 in. This inner diameter allows compatibility with a 12 F catheter. In some embodiments, the inner lumen diameter is about 0.2-0.3 in. This inner diameter allows compatibility with a 20 F catheter. In some embodiments, the rigidizing catheter inner lumen is about 0.34 in. This inner diameter allows compatibility with a 26 F catheter.

In some embodiments, the rigidizing catheter outer diameter is about 0.2-0.4 in. In some embodiments, the outer diameter is about 0.23 in. or about 18 F. In some embodiments, the outer diameter is about 0.34 in. or about 26 F. In some embodiments, the rigidizing catheter as a length of about 70-120 cm (or about 80-115 cm, or about 85-115 cm, etc.). In some embodiments, the rigidizing catheter has a minimum bend radius of about 1-2 in. (or about 1.5 in.).

The system can include an obturator 1084 which can be used during navigation of the rigidizing catheter 1030. Examples of obturators are described in International Application No. PCT/US2023/062206, filed Feb. 6, 2023, the entirety of which is incorporated by reference herein. The obturator 1084 can comprise a connector 1062 at its proximal end. The obturator 1084 may be configured to be inserted into the rigidizing catheter 1030 through the hemostatic seal. Once the obturator 1084 is completely inserted within the rigidizing catheter 1030, the obturator can be rotated to lock it in place with respect to the hemostatic seal. The rotation of connector 1062 relative to a threaded connection 1064 on the hemostatic seal region may create a lock between the mating mechanism 1064 and corresponding mating mechanism 1066 (e.g., thread, bayonet connection, etc.) on the obturator connector 1062. In some embodiments, the connector and obturator are rotated about 90° relative to one another. Other amounts of relative rotation (e.g., about 30-360° are also contemplated).

As described in further details below, the rigidizing catheter can be transitioned between a flexible and a rigid state upon application of pressure. In the flexible state, the rigidizing catheter can be navigated (in some examples over a guidewire, though in some examples no guidewire is needed or used) through tortuous anatomy and vasculature. Once the rigidizing catheter has navigated to a desired location, transitioning the rigidizing catheter to a rigid state preserves the shape of the catheter at the time of rigidization and provides a stable pathway for navigation of other accessories and tools through the catheter, again without requiring a guidewire (or without further use of a guidewire). The ability to transform the rigidizing catheter (also referred to herein as a rigidizing aspiration sheath) provides a number of advantages to the apparatuses described herein that are not realizable with existing apparatuses. In particular, in the flexible configuration of the aspiration sheath may conform to the patient's anatomy, whereas a standard catheter is more rigid and therefore exerts a force that forces the anatomy to adapt to it; once converted to a rigid (or semi-rigid) state, the aspiration sheath may prevent or minimize force on the same anatomy, and may support operations from the aspiration sheath.

In some embodiments, the rigidizing catheter can be configured to be at maximum rigidization with the application of about 6 atm of positive pressure. In some applications, maximum rigidization can occur with the application of about 10, 20, 30, 40, or 50 atm. In some embodiments, the rigidizing catheter can be configured to be flexible upon application of negative pressure. In some embodiments, the rigidizing catheter (e.g., rigidizing aspiration sheath catheter) can be configured to be flexible without the application of pressure. Other configurations are also contemplated, as described in further detail below.

Any dynamically rigidizing structure may be used as part of the rigidizing catheter described herein, or optionally the aspiration catheters. For example, variations of rigidizing catheters that can be used with the aspiration system described herein can be long, thin, and hollow and can transition quickly from a flexible configuration (i.e., one that is relaxed, limp, or floppy) to a rigid configuration (i.e., one that is stiff and/or holds the shape it is in when it is rigidized). In some examples the rigidizing apparatus may include a plurality of layers (e.g., coiled or reinforced layers, slip layers, rigidizing layers, bladder layers, sealing sheaths, etc.) that can together form the wall of the rigidizing devices, which may be referred to as “layered rigidizing apparatuses.” Unless the context makes clear otherwise, the methods and apparatuses described herein may refer to any appropriate rigidizing device, including layered rigidizing apparatuses. For example, the rigidizing devices (members, apparatuses, etc.) described herein may be rigidized by jamming particles, by phase change, by interlocking components (e.g., cables with discs or cones, etc.) or any other rigidizing mechanism. The rigidizing devices can transition from the flexible configuration to the rigid configuration, for example, by applying a vacuum or pressure to the wall of the rigidizing device or within the wall of the rigidizing device. With the vacuum or pressure removed, the layers can easily shear or move relative to each other, including the rigidizing layer(s). With the vacuum or pressure applied, the layers can transition to a condition in which they exhibit substantially enhanced ability to resist shear, movement, bending, torque and buckling, thereby providing system rigidization. Examples of rigidizing layers that may be rigidized by the application of pressure (positive or negative pressure), may include layers formed of one or more filaments that cross over each other, e.g., in a braid, weave, knit, etc. The rigidizing layer may be organized (e.g., knit, braid, weave, etc.) or may be disorganized (e.g., a mixture of filament fragments of uniform or varying lengths), or some combination of these. The crossing filaments of the rigidizing layer may be adjacent to the bladder layer, which may be driven against the rigidizing layer to rigidize the device. The examples of rigidizing apparatuses described herein may use pressure (positive pressure) and/or negative pressure to selectively and controllably rigidize the apparatus. In general, the method described herein may be used with any appropriate rigidizing apparatus.

The rigidizing (e.g., selectively rigidizing) apparatuses described herein can provide rigidization for a variety of medical applications, including catheters, sheaths, scopes (e.g., endoscopes), wires, overtubes, trocars or laparoscopic instruments. The rigidizing devices can function as a separate add-on device or can be integrated into the body of catheters, sheaths, scopes, wires, or laparoscopic instruments. The devices described herein can also provide rigidization for non-medical structures.

Any of the rigidizing apparatuses described herein may include a rigidizing length having a wall (e.g., tubular wall) with a plurality of layers, which may be tubular layers, including a rigidizing layer, an outer layer (part of which is cut away in this example to show the braid thereunder), and an inner layer. The apparatus may include a handle having a vacuum or pressure inlet to supply vacuum or pressure to the rigidizing device. A separate flush/perfusion inlet and line may also be included, e.g., for applying fluid (e.g., saline, contrast, etc.) through the lumen of the rigidizing catheter. An actuation element can be used to turn the vacuum or pressure on and off to thereby transition the rigidizing device between flexible and rigid configurations. The distal tip of the rigidizing catheter can be smooth, flexible, and atraumatic to facilitate distal movement of the rigidizing device through the body. Further, the tip can taper from the distal end to the proximal end to further facilitate distal movement of the rigidizing device through the body. The rigidizing apparatus may be configured as an outer rigidizing catheter, but other configurations may be used.

As the rigidizing device is rigidized, it locks into the shape it was in before positive and/or negative pressure was applied, i.e., it does not straighten, bend, or otherwise substantially modify its shape (e.g., it may stiffen in a looped configuration or in a serpentine shape). The stiffening effect on the inner or outer layers (e.g., made of coil-wound tube) can be a small percentage (e.g., 5%) of the maximum load capability of the rigidizing device in bending, thereby allowing the rigidizing device to resist straightening. Upon release of the negative and/or positive pressure, length of filaments (e.g., braids or strands) within the rigidizing layer can unlock relative to one another and again move relative to each other (e.g., slide over each other) so as to allow bending of the rigidizing device. As the rigidizing apparatus is made more flexible through the release of positive and/or negative pressure, it may at least initially maintain the shape it was in before the positive and/or negative pressure was released. However, as described herein, in some cases, the rigidizing apparatus (e.g., the outer rigidizing catheter) may be configured so that, upon gradual release of the pressure maintaining the apparatus in the rigid state, may relax into a different shape, allowing steering of the distal end region of the apparatus. This is different from previously described apparatuses, including pressure-rigidizing apparatuses, in which changing the pressure to convert the device into a flexible configuration did not result in straighten, bending, or otherwise substantially modifying its shape. Thus, apparatuses described herein can transition be steered by controlling the transition from the rigid configuration to a more flexible, less-stiff configuration.

The rigidizing apparatuses (e.g., rigidizing catheter, and in some case aspiration catheters) described herein can toggle between a rigid configuration and a flexible configuration at a controlled rate, and in some examples with an indefinite number of transition cycles. In some examples the degree of rigidization (e.g., the stiffness) of the apparatus may also be adjusted, for example, by adjusting the positive pressure (in examples that are rigidized by positive pressure) or negative pressure (in examples rigidized by vacuum). As interventional medical devices are made longer and inserted deeper into the human body, and as they are expected to do more exacting therapeutic procedures, there is an increased need for precision and control. Selectively rigidizing devices (including selectively rigidizing overtubes) as described herein can advantageously provide both the benefits of flexibility (when needed) and the benefits of stiffness (when needed). Further, the rigidizing devices described herein can be used, for example, with classic endoscopes, colonoscopes, robotic systems, and/or navigation systems, such as those described in International Patent Application No. PCT/US2016/050290, filed Sep. 2, 2016, titled “DEVICE FOR ENDOSCOPIC ADVANCEMENT THROUGH THE SMALL INTESTINE,” the entirety of which is incorporated by referenced herein.

The rigidizing catheters described herein can additionally or alternatively include any of the features described with respect to International Patent Application No. PCT/US2016/050290, filed on Sep. 2, 2016, titled “DEVICE FOR ENDOSCOPIC ADVANCEMENT THROUGH THE SMALL INTESTINE,” published as WO 2017/041052, International Patent Application No. PCT/US2018/042946, filed on Jul. 19, 2018, titled “DYNAMICALLY RIGIDIZING OVERTUBE,” published as WO 2019/018682, International Patent Application No. PCT/US2019/042650, filed on Jul. 19, 2019, titled “DYNAMICALLY RIGIDIZING COMPOSITE MEDICAL STRUCTURES,” published as WO 2020/018934, and International Patent Application No. PCT/US2020/013937 filed on Jan. 16, 2020, titled “DYNAMICALLY RIGIDIZING COMPOSITE MEDICAL STRUCTURES,” the entireties of which are incorporated by reference herein.

The rigidizing devices described herein can be provided in multiple configurations, including different lengths and diameters. In some examples, the rigidizing devices can include working channels (for instance, for allowing the passage of typical endoscopic tools within the body of the rigidizing device), balloons, nested elements, and/or side-loading features.

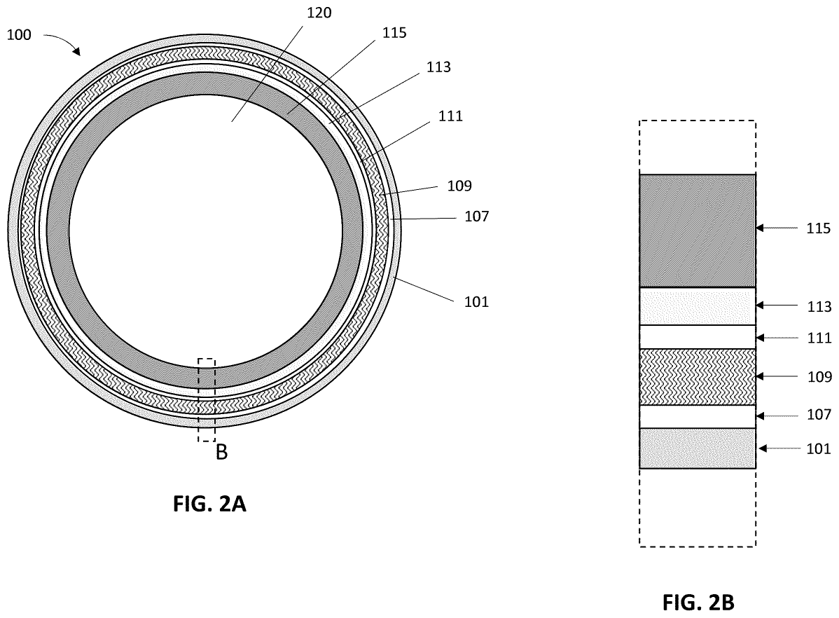

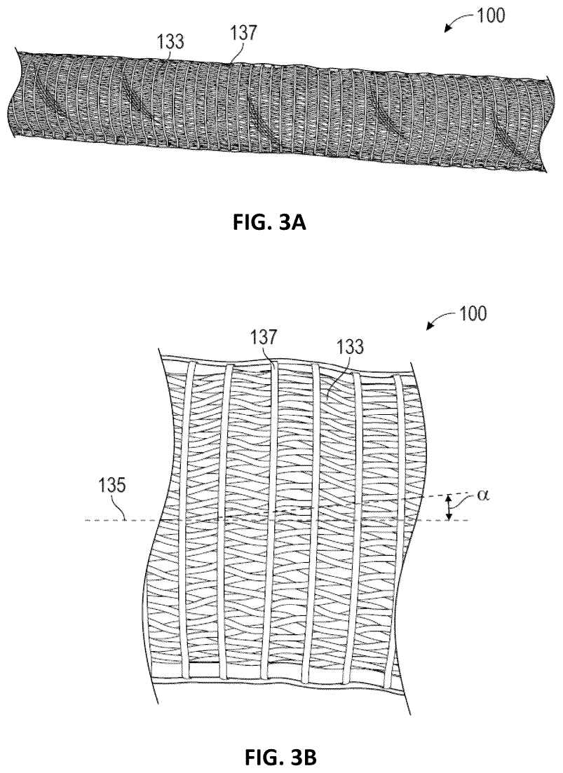

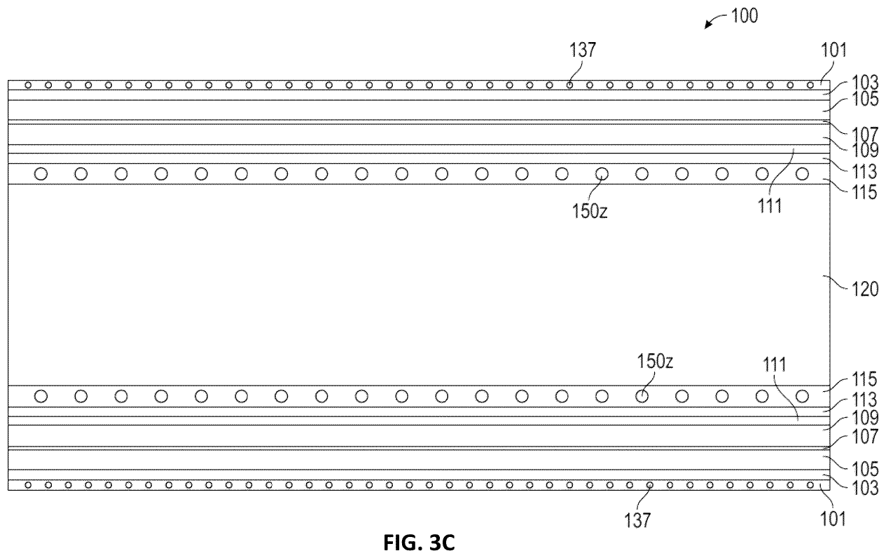

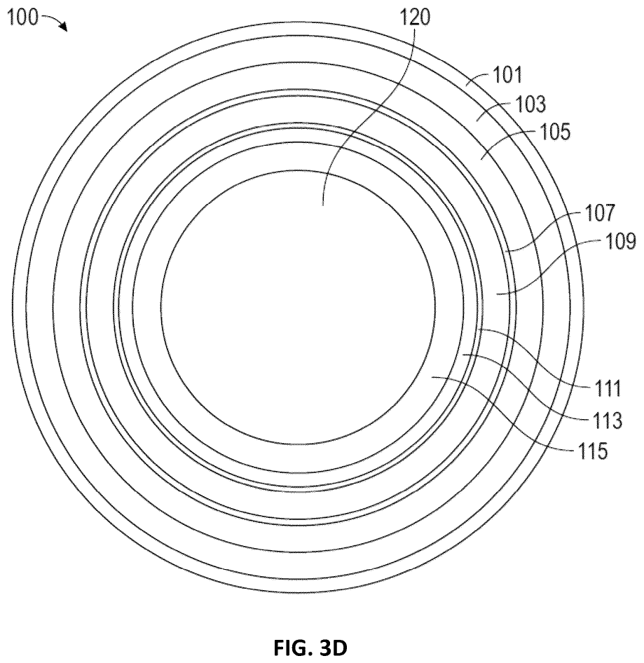

For example, a rigidizing apparatus 100 (also referred to as an apparatus, e.g., system and/or device, including a rigidizable member) may be configured to be rigidized by the application of vacuum, e.g., negative pressure. These apparatuses may generally be formed of layers that are configured to form a laminates structure when negative pressure is applied, so that one or more braided or woven layers may be reversibly fused to a flexible outer layer that is driven against a more rigid inner layer. FIGS. 2A-2B illustrate (not to scale) one example of a section through a rigidizing member of an apparatus (e.g., device, system) that is rigidized by the application of vacuum. FIG. 3B shows an enlarged view of the arrangement of the layers of FIG. 2A in the un-rigidized configuration. In this example, the rigidizable member includes an innermost layer 115 that is configured to provide an inner surface against which the remaining layers can be consolidated (e.g., when vacuum is applied). The innermost layer 115 can include a reinforcement element, e.g., coil. The rigidizing member may also include an optional slip layer 113 over (e.g., radially outwards of) the innermost layer. The slip layer may be, e.g., a lubrication, coating and/or powder (e.g., talcum powder) on the outer surface of the inner layer 115 and/or within the gap layer 111. A radial gap layer 111 may separate the slip layer 113 from a rigidizing layer 109 (which in some examples is a knitted, braided or woven layer), providing a space between the rigidizing layer and the slip layer for the braided layer(s) thereover to move within, e.g., when no vacuum is applied; this space or gap may be removed when vacuum is applied, allowing the braided or woven layer(s) to move radially inward upon application of vacuum. A second gap layer 107 may be present between the rigidizing layer 109 and may be similar to layer 111. As will be described in reference to FIGS. 3A-3D, multiple rigidizing layers may be included (e.g., 2, 3 4 or more rigidizing layers may be included) and may be separated by additional gap layers and/or slip layers. The outermost layer 101 can be separated from the rigidizing layer(s) by a gap layer and can be configured to move radially inward when a vacuum is applied to pull down against the rigidizing layer(s) and conform onto the surface(s) thereof. The outermost layer 101 can be soft and atraumatic and can be sealed at both ends to create a vacuum-tight chamber with the innermost layer 115. The outermost layer 101 can be elastomeric, e.g., made of urethane. The hardness of the outermost layer 101 can be, for example, 30 A to 80 A (Shore A durometer scale). Further, the outermost layer 101 can have a thickness of 0.0001-0.01″, such as approximately 0.001″, 0.002, 0.003″ or 0.004″. Alternatively, the outermost layer can be a plastomer. Alternatively, the outermost layer can be a plastic, including, for example, LDPE, nylon, or PEEK.

FIGS. 3A-3D illustrate an example of a tubular rigidizing member of an apparatus 100 that includes multiple rigidizing layers. As in FIGS. 2A-2B, the apparatus includes a tube having a wall formed of a plurality of layers positioned around a lumen 120 (e.g., for placement of an instrument or endoscope therethrough). A vacuum can be supplied between the layers to rigidize the rigidizing device 100. Any of the tubular apparatuses described herein may instead include a solid core forming the inner layer 115.

The innermost layer 115 can be configured to provide an inner surface against which the remaining layers can be consolidated, for example, when a vacuum is applied within the walls of the rigidizing device 100. The structure can be configured to minimize bend force and/or maximize flexibility in the non-vacuum condition. In some examples, the innermost layer 115 can include a reinforcement element 150z or coil within a matrix, as described above. In the example shown in FIG. 3C, the layer 113 over (i.e., radially outwards of) the innermost layer 115 can be a slip layer. The layer 111 can be a radial gap (i.e., a space). The gap layer 111 can provide space for the braided layer(s) thereover to move within (when no vacuum is applied) as well as space within which the braided or woven layers can move radially inward (upon application of vacuum).

The layer 109 can be a first rigidizing layer including braided strands 133 similar to as described elsewhere herein. The rigidizing layer can be, for example, 0.001″ to 0.040″ thick. For example, a rigidizing layer can be 0.001″, 0.003″, 0.005″, 0.010″, 0.015″, 0.020″, 0.025″ or 0.030″ thick. In some examples, as shown in FIG. 3B, the braid can have tensile or hoop fibers 137. Hoop fibers 137 can be spiraled and/or woven into a rigidizing layer. Further, the hoop fibers 137 can be positioned at 2-50, e.g., 20-40 hoops per inch. The hoop fibers 137 can advantageously deliver high compression stiffness (to resist buckling or bowing out) in the radial direction but can remain compliant in the direction of the longitudinal axis 135 of the rigidizing device 100. That is, if axial compression is applied to the rigidizing device 100, the rigidizing layer 109 will try to expand in diameter as it compresses. The hoop fibers 137 can resist this diametrical expansion. Accordingly, the hoop fiber 137 can provide a system that is flexible in bending but still resists both tension and compression.

The layer 107 can be another radial gap layer similar to layer 111.

In some examples, the rigidizing devices described herein can have more than one rigidizing layer. For example, the rigidizing devices can include two, three, or four rigidizing layers. Referring to FIG. 3C, the layer 105 can be a second rigidizing layer 105. The second rigidizing layer 105 can have any of the characteristics described with respect to the first rigidizing layer 109. In some examples, the braid of second rigidizing layer 105 can be identical to the braid of first rigidizing layer 109. In other examples, the braid of second rigidizing layer 105 can be different than the braid of the first rigidizing layer 109. For example, the braid of the second rigidizing layer 105 can include fewer strands and have a larger braid angle α than the braid of the first rigidizing layer 109. Having fewer strands can help increase the flexibility of the rigidizing device 100 (relative to having a second strand with equivalent or greater number of strands), and a larger braid angle α can help constrict the diameter of the of the first rigidizing layer 109 (for instance, if the first rigidizing layer is compressed) while increasing/maintaining the flexibility of the rigidizing device 100. As another example, the braid of the second rigidizing layer 105 can include more strands and have a larger braid angle α than the braid of the first rigidizing layer 109. Having more strands can result in a relatively tough and smooth layer while having a larger braid angle α can help constrict the diameter of the first rigidizing layer 109.

The layer 103 can be another radial gap layer similar to layer 111. The gap layer 103 can have a thickness of 0.0002-0.04″, such as approximately 0.03″. A thickness within this range can ensure that the strands 133 of the rigidizing layer(s) can easily slip and/or bulge relative to one another to ensure flexibility during bending of the rigidizing device 100.

The outermost layer 101 can be configured to move radially inward when a vacuum is applied to pull down against the rigidizing layers 105, 109 and conform onto the surface(s) thereof. The outermost layer 101 can be soft and atraumatic and can be sealed at both ends to create a vacuum-tight chamber with layer 115. The outermost layer 101 can be plastic, a plastomer, or elastomeric, e.g., made of urethane. The hardness of the outermost layer 101 can be, for example, 30 A to 80 A. Further, the outermost layer 101 can have a thickness of 0.0001-0.01″, such as approximately 0.001″, 0.002, 0.003″ or 0.004″. Alternatively, the outermost layer can be plastic, including, for example, LDPE, nylon, or PEEK.

In some examples, the outermost layer 101 can, for example, have tensile or hoop fibers 137 extending therethrough. The hoop fibers 137 can be made, for example, of aramids (e.g., Technora, nylon, Kevlar), Vectran, Dyneema, carbon fiber, fiber glass, boron, basalt, polyester, or other plastic. Further, the hoop fibers 137 can be positioned at 2-50, e.g., 20-40 hoops per inch. In some examples, the hoop fibers 137 can be laminated within an elastomeric sheath. The hoop fibers can advantageously deliver higher stiffness in one direction compared to another (e.g., can be very stiff in the hoop direction, but very compliant in the direction of the longitudinal axis of the rigidizing device). Additionally, the hoop fibers can advantageously provide low hoop stiffness until the fibers are placed under a tensile load, at which point the hoop fibers can suddenly exhibit high hoop stiffness.

In some examples, the outermost layer 101 can include a lubrication, coating and/or powder (e.g., talcum powder) on the outer surface thereof to improve sliding of the rigidizing device through the anatomy. The coating can be hydrophilic (e.g., a Hydromer® coating or a Surmodics® coating) or hydrophobic (e.g., a fluoropolymer). The coating can be applied, for example, by dipping, painting, or spraying the coating thereon.

The innermost layer 115 can similarly include a lubrication, coating (e.g., hydrophilic or hydrophobic coating), and/or powder (e.g., talcum powder) on the inner surface thereof configured to allow the bordering layers to more easily shear relative to each other, particularly when no vacuum is applied to the rigidizing device 100, to maximize flexibility.

In some examples, the outermost layer 101 can be loose over the radially inward layers. For instance, the inside diameter of layer 101 (assuming it constitutes a tube) may have a diametrical gap of 0″-0.200″ with the next layer radially inwards (e.g., with a rigidizing layer). This may give the vacuum rigidized system more flexibility when not under vacuum while still preserving a high rigidization multiple. In other examples, the outermost layer 101 may be stretched some over the next layer radially inwards (e.g., the rigidizing layer). For instance, the zero-strain diameter of a tube constituting layer 101 may be from 0-0.200″ smaller in diameter than the next layer radially inwards and then stretched thereover. When not under vacuum, this system may have less flexibility than one wherein the outer layer 101 is looser. However, it may also have a smoother outer appearance and be less likely to tear during use.

In some examples, the outermost layer 101 can be loose over the radially inward layers. A small positive pressure may be applied underneath the layer 101 in order to gently expand layer 101 and allow the rigidizing device to bend more freely in the flexible configuration. In this example, the outermost layer 101 can be elastomeric and can maintain a compressive force over the braid, thereby imparting stiffness. Once positive pressure is supplied (enough to nominally expand the sheath off of the braid, for example, 2 psi), the outermost layer 101 is no longer a contributor to stiffness, which can enhance baseline flexibility. Once rigidization is desired, positive pressure can be replaced by negative pressure (vacuum) to deliver stiffness.

A vacuum can be carried within rigidizing device 100 from minimal to full atmospheric vacuum (e.g., approximately 14.7 psi). In some examples, there can be a bleed valve, regulator, or pump control such that vacuum is bled down to any intermediate level to provide a variable stiffness capability. The vacuum pressure can advantageously be used to rigidize the rigidizing device structure by compressing the layer(s) of braided sleeve against neighboring layers. Braid is naturally flexible in bending (i.e., when bent normal to its longitudinal axis), and the lattice structure formed by the interlaced strands distort as the sleeve is bent in order for the braid to conform to the bent shape while resting on the inner layers. This results in lattice geometries where the corner angles of each lattice element change as the braided sleeve bends. When compressed between conformal materials, such as the layers described herein, the lattice elements become locked at their current angles and have enhanced capability to resist deformation upon application of vacuum, thereby rigidizing the entire structure in bending when vacuum is applied. Further, in some examples, the hoop fibers through or over the braid can carry tensile loads that help to prevent local buckling of the braid at high applied bending load.

The stiffness of the rigidizing device 100 can increase from 2-fold to over 50-fold, for instance 10-fold, 15-fold, or 20-fold, when transitioned from the flexible configuration to the rigid configuration. In one specific example, the stiffness of a rigidizing device similar to rigidizing device 100 was tested. The wall thickness of the test rigidizing device was 1.0 mm, the outer diameter was 17 mm, and a force was applied at the end of a 9.5 cm long cantilevered portion of the rigidizing device until the rigidizing device deflected 10 degrees. The forced required to do so when in flexible mode was only 30 grams while the forced required to do so in rigid (vacuum) mode was 350 grams.

In some examples of a vacuum rigidizing device 100, there can be only one rigidizing layer. In other examples of a vacuum rigidizing device 100, there can be two, three, or more rigidizing layers. In some examples, one or more of the radial gap layers or slip layers of rigidizing device 100 can be removed. In some examples, some or all of the slip layers of the rigidizing device 100 can be removed.

The rigidizing layers described herein can act as a variable stiffness layer. The variable stiffness layer can include one or more variable stiffness elements or structures that, when activated (e.g., when vacuum is applied), the bending stiffness and/or shear resistance is increased, resulting in higher rigidity. In some of the apparatuses described herein, the variable stiffness layer comprises one or more filaments forming a plurality of filament regions that cross over each other and are free to move (e.g., slide) relative to one another in the flexible configuration, but may be increasingly constrained in the rigidizing state(s) when applying pressure (positive and/or negative pressure), which in some examples may drive a bladder layer against and/or into the variable stiffness layer. Other variable stiffness elements can be used in addition to or in place of the rigidizing layer. In some examples, engagers can be used as a variable stiffness element, as described in International Patent Application No. PCT/US2018/042946, filed Jul. 19, 2018, titled “DYNAMICALLY RIGIDIZING OVERTUBE,” the entirety of which is incorporated by reference herein. Alternatively or additionally, the variable stiffness element can include particles or granules, jamming layers, scales, rigidizing axial members, rigidizers, longitudinal members or substantially longitudinal members.

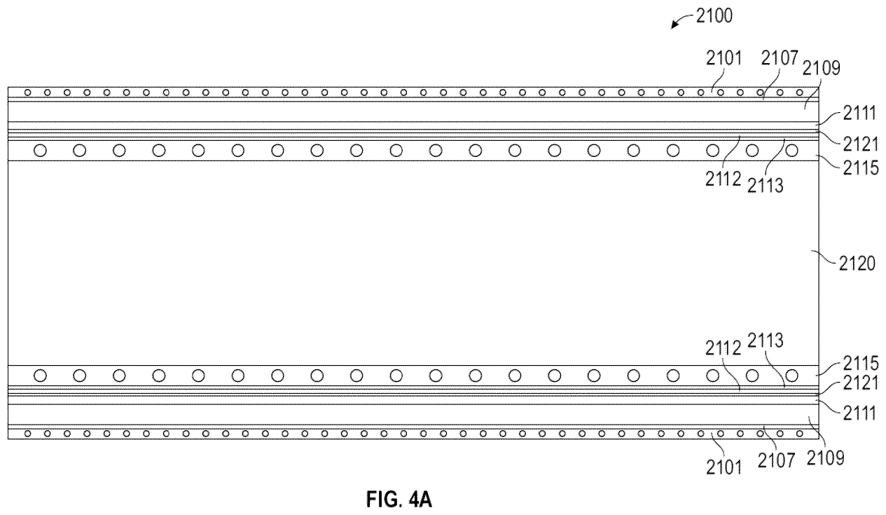

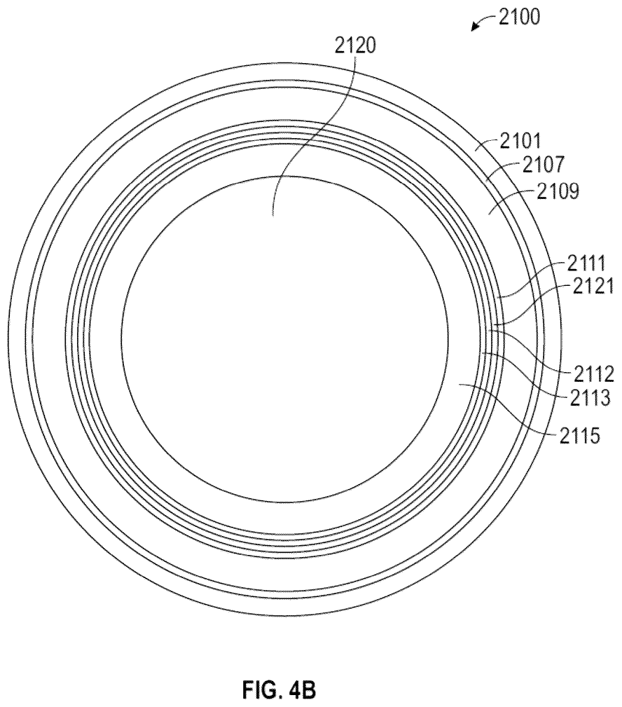

The rigidizable apparatuses described herein may also be rigidized by the application of positive pressure, rather than vacuum. For example, referring to FIGS. 4A-4B, the rigidizing apparatus (e.g., device or system) 2100 can be similar to rigidizing apparatus 100 described above, except that it can be configured to hold pressure (e.g., of greater than 1 atm) therein for rigidization rather than vacuum. A pressure-activated rigidizing device 2100 can also include a plurality of layers positioned around a lumen 2120 (e.g., for placement of an instrument or endoscope therethrough).

For example, FIGS. 4A-4B illustrate (not to scale) longitudinal and radial sections through an example of a pressure-activated rigidizable member of a rigidizing apparatus. The rigidizing device 2100 shown in FIGS. 4A and 4B can include an innermost layer 2115 (similar to innermost layer 115), a slip layer 2113 (similar to slip layer 113), a pressure gap 2112, a bladder layer 2121, a gap layer 2111 (similar to gap layer 111), a rigidizing layer 2109 (similar to rigidizing layer 109) or other variable stiffness layer as described herein, a gap layer 2107 (similar to layer 107), and an outermost containment layer 2101.

The pressure gap 2112 can be a sealed chamber that provides a gap for the application of pressure to layers of rigidizing device 2100. The pressure can be supplied to the pressure gap 2112 using a fluid or gas inflation/pressure media. The inflation/pressure media can be water or saline solution or, for example, a lubricating fluid such as oil or glycerin. The lubricating fluid can, for example, help the layers of the rigidizing device 2100 flow over one another in the flexible configuration. The inflation/pressure media can be supplied to the gap 2112 during rigidization of the rigidizing device 2100 and can be partially or fully evacuated therefrom to transform the rigidizing device 2100 back to the flexible configuration. In some examples, the pressure gap 2112 of the rigidizing device 2100 can be connected to a pre-filled pressure source, such as a pre-filled syringe or a pre-filled insufflator, thereby reducing the physician's required set-up time.

The bladder layer 2121 can be made, for example, of a low durometer elastomer (e.g., of shore 20 A to 70 A) or a thin plastic sheet. The bladder layer 2121 can be formed out of a thin sheet of plastic or rubber that has been sealed lengthwise to form a tube. The lengthwise seal can be, for instance, a butt or lap joint. For instance, a lap joint can be formed in a lengthwise fashion in a sheet of rubber by melting the rubber at the lap joint or by using an adhesive. In some examples, the bladder layer 2121 can be 0.0002-0.020″ thick, such as approximately 0.005″ thick. The bladder layer 2121 can be soft, high-friction, stretchy, and/or able to wrinkle easily. In some examples, the bladder layer 2121 is a polyolefin or a PET. The bladder 2121 can be formed, for example, by using methods used to form heat shrink tubing, such as extrusion of a base material and then wall thinning with heat, pressure and/or radiation. When pressure is supplied through the pressure gap 2112, the bladder layer 2121 can expand through the gap layer 2111 to push the rigidizing layer 2109 against the outermost containment layer 2101 such that the relative motion of the braid strands is reduced.

The outermost containment layer 2101 can be a tube, such as an extruded tube. Alternatively, the outermost containment layer 2101 can be a tube in which a reinforcing member (for example, metal wire, including round or rectangular cross-sections) is encapsulated within an elastomeric matrix, similar to as described with respect to the innermost layer for other examples described herein. In some examples, the outermost containment layer 2101 can include a helical spring (e.g., made of circular or flat wire), and/or a tubular braid (such as one made from round or flat metal wire) and a thin elastomeric sheet that is not bonded to the other elements in the layer. The outermost containment layer 2101 can be a tubular structure with a continuous and smooth surface. This can facilitate an outer member that slides against it in close proximity and with locally high contact loads (e.g., a nested configuration as described further herein). Further, the outer layer 2101 can be configured to support compressive loads, such as pinching. Additionally, the outer layer 2101 (e.g., with a reinforcement element therein) can be configured to prevent the rigidizing device 2100 from changing diameter even when pressure is applied.

Because both the outer layer 2101 and the inner layer 2115 include reinforcement elements therein, the rigidizing layer 2109 can be reasonably constrained from both shrinking diameter (under tensile loads) and growing in diameter (under compression loads).

By using positive pressure rather than vacuum to transition from the flexible state to the rigid state, the rigidity of the rigidizing device 2100 can be increased. For example, in some examples, the pressure supplied to the pressure gap 2112 can be between 1 and 40 atmospheres, such as between 2 and 40 atmospheres, such as between 4 and 20 atmospheres, such as between 5 and 10 atmospheres. In some examples, the pressure supplied is approximately 2 atm, approximately 4 atmospheres, approximately 5 atmospheres, approximately 10 atmospheres, approximately 20 atmospheres. In some examples, the rigidizing device 2100 can exhibit change in relative bending stiffness (as measured in a simple cantilevered configuration) from the flexible configuration to the rigid configuration of 2-100 times, such as 10-80 times, such as 20-50 times. For example, the rigidizing device 2100 can have a change in relative bending stiffness from the flexible configuration to the rigid configuration of approximately 10, 15, 20, or 25, 30, 40, 50, or over 100 times.

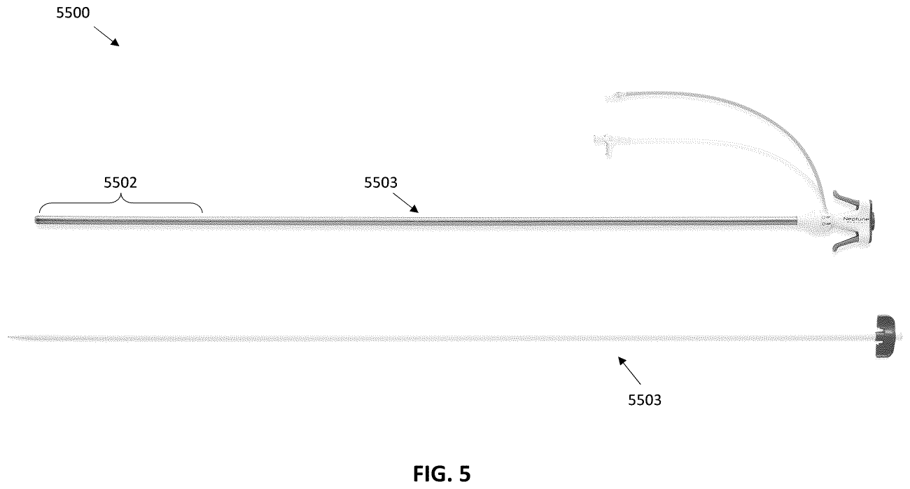

Any of the rigidizing devices described herein can have a distal end section or sections with a different design that the main elongate body of the rigidizing device. FIG. 5 is another view of an outer rigidizing catheter apparatus 5500 that can have a main elongate body 5503 and a distal end section 5502. The distal end section 5502 can be rigidized as part of the main elongate body 5503 (e.g., by vacuum and/or positive pressure).

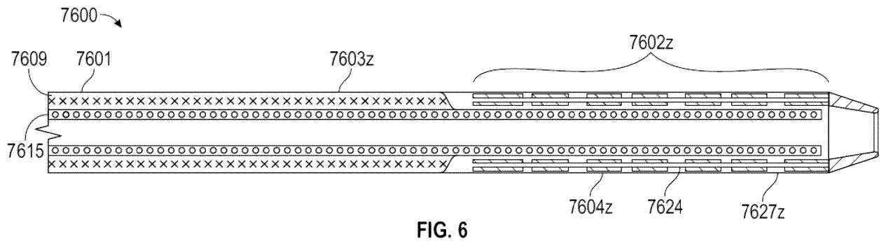

In some examples (e.g., shown schematically and not to scale in FIG. 6) the distal end section 7602z can include a plurality of linkages 7604z that are actively controlled, such as via cables 7624, for steering of the rigidizing device 7600. The device 7600 may be similar to device 5500 except that it includes cables 7624 configured to control movement of the device. Cables can extend therethrough in any manner, e.g., for manually steering. However in some cases it may be beneficial to steer the outer rigidizing catheter without using or including cables, which may take up space and add complexity.

FIG. 7 shows a section through one example of a proximal end region of a rigidizing catheter 1130 showing a hemostatic valve region 1139 including a valve body 1131 and a pair of levers 1142, 1142′ for controlling opening and sealing the hemostasis valve of the rigidizing catheter either closed or sealed over a device within the lumen of the rigidizing catheter.

In general, any of the apparatuses described herein may include a flush port. The flush port may be particularly advantageous, as it may allow for flushing of the catheter, and may also be used to inject contrast and for continuous hemodynamic measurements.

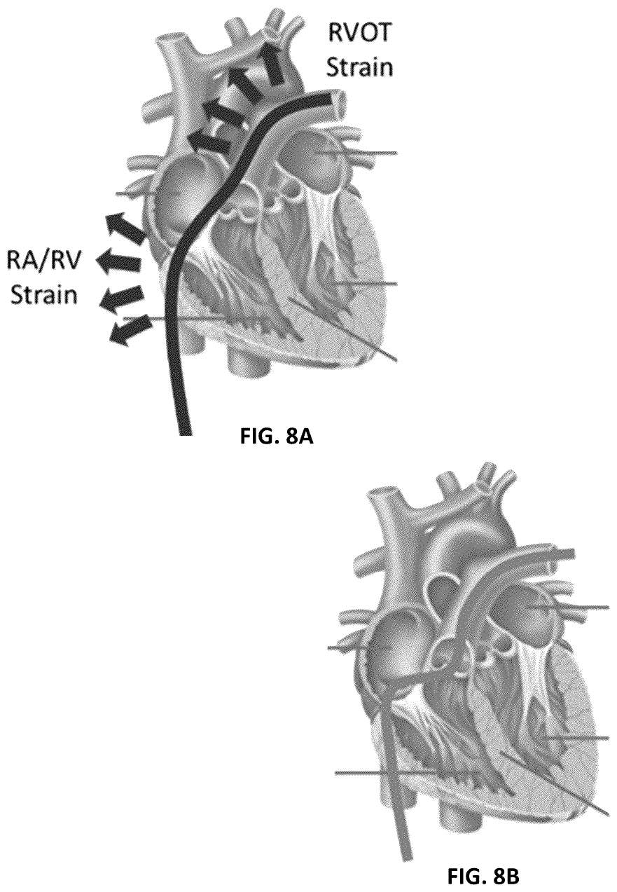

FIGS. 8A and 8B show a traditional catheter navigated to the pulmonary artery (FIG. 8A) and the rigidizing system described herein navigated to the pulmonary artery (FIG. 8B). As shown in FIGS. 8A and 8B, the rigidizing system's ability to establish the pathway in a flexible state leads to reduced strain and reduced potential for hemodynamic compromise and injury.

As mentioned above, any of the rigidizing catheters described herein may be steered by changing the pressure within in rigidizing the catheters. In general, the outer rigidizing catheter may be initially fixed and rigidized, e.g., in a curved or bent configuration; in some case the outer rigidizing catheter may be rigidized in a tight configuration by the application of positive and/or negative pressure. The outer rigidizing catheter may be biased, e.g., based on the construction of the catheter, and/or based on a tool or device coupled to the outer rigidizing catheter, such as an obturator within the outer rigidizing catheter, so that when the outer rigidizing catheter is de-rigidized, e.g., by changing the pressure (positive and/or negative pressure) applied, the outer rigidizing catheter may change the tip position as it relaxed towards the bias configuration. The rate of change of the tip region may be based on the rate of change in the pressure. In some cases the tip region of the outer rigidizing catheter may be moved by releasing the pressure to change the outer rigidizing catheter from the rigid configuration to the more flexibility configuration; for example the distal end region of the outer rigidizing catheter may straighten out, and the orientation of the tip region may change as it straightens out. In general, during this process of straightening out, the user (e.g., doctor, physician, medical technician, nurse, etc.) can “freeze” the orientation of the catheter tip into the desired direction by applying pressure (positive and/or negative), pressurizing the outer rigidizing catheter and fixing the outer rigidizing catheter again. The pressure may again be released to allow the movement of the tip region to continue as the outer rigidizing catheter relaxes towards the bias shape. Thus, these steps can be repeated as many times as needed, until the catheter tip region has fully moved (e.g., in some cases fully straightened out) and will cease to move further. The movement of the tip region can be encouraged by cycling between the rigid and the flexible configuration. In any of these example, the application and release of pressure does not drive movement of the catheter tip region, but allows the tip region to move based on the bias force, e.g., due to the structure of the outer rigidizing catheter itself and/or a tool within the outer rigidizing catheter.

As mentioned, the speed of the movement of the catheter tip direction change can be controlled by how quickly the catheter is de-pressurized and therefore allowed to relax.

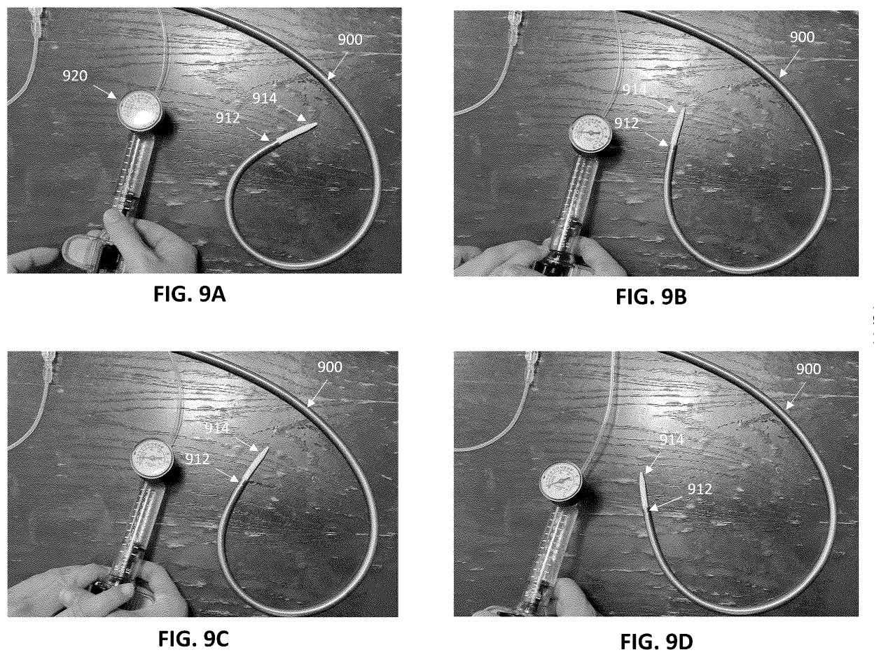

FIGS. 9A-9D illustrate one example of an outer rigidizing catheter 900 that is initially shown in a tight curve or bend at the distal end region 912. In FIG. 9A the outer rigidizing catheter 900 is in a rigid configuration, and an obturator 914 is inserted through the outer rigidizing catheter. In this example a positive pressure has been applied from the pressure controller 920 and the pressure is maintained to keep the outer rigidizing catheter in a rigid configuration. FIG. 9B shows the outer rigidizing catheter 900 after the pressure has been gradually released (e.g., at a rate of, e.g., between 10-100 mmHg/sec, such as about 50 mmHg/sec) to allow the apparatus distal tip region 912 to slowly move from the initial position in FIG. 9A to a second position shown in FIG. 9B. At any time during the movement the user may stop the release of pressure and may re-apply pressure to re-rigidize the outer rigidizing catheter holding the instantaneous position of the distal tip region. The distal tip region may then be moved again by again gradually (or at a slow, controlled rate) releasing the pressure, as shown in FIG. 9C. in this example, the tip 912 again slowly moves, as shown. This process of rigidizing and de-rigidizing may be repeated multiple times, as shown in FIG. 9D, to reposition the tip of the outer rigidizing catheter without requiring guidewire. Although the example shown in FIGS. 9A-9D includes an obturator within the outer rigidizing catheter, in some cases the outer rigidizing catheter does not include an obturator or any other tool.

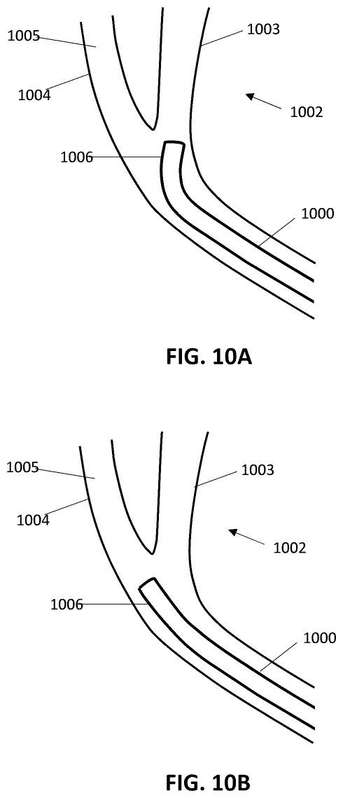

FIGS. 10A and 10B show an example of steering a rigidizing catheter 1000 by adjusting pressure. FIG. 10A shows the rigidizing catheter 1000 after being advanced within a vasculature 1002 toward a branched region of the vasculature 1002. The catheter 1000 may be rigidized by applying and maintaining pressure (negative or positive) within the walls of the catheter 1000. The amount of pressure supplied to the catheter 1000 to sufficiently rigidize the catheter 1000 for guiding an instrument (e.g., aspirating catheter) therein may vary. In some examples, as discussed above, a positive pressure may range between 1 and 40 atmospheres.

In the example of FIG. 10A, a distal end region 1006 of the catheter 1000 is fixed in a tight curved shape. However, it would be desirable to steer the distal end region 1006 toward a target region 1005 that is within in a first vessel 1004 that branches a second vessel 1003. In one example, the first vessel 1004 may be the left pulmonary artery and the second vessel 1003 may be the right pulmonary artery. If the rigidizing catheter 1000 is being used to aspirate blood clots, the target region 1005 may be identified as a region of the vasculature 1002 that is identified as having one or more blood clots. As shown in FIG. 10A, a distal end section/region 1006 of the catheter 1000 is pointed in a direction that is not toward the target region 1005.

To steer the distal end region 1006 toward the target region 1005, the rigidizing pressure that is within the walls of the catheter 1000 to maintain its rigidity may be gradually changed (e.g., released) until the distal end region 1006 relaxes to a preferred shape, such as shown in FIG. 10B. For example, if the catheter 1000 is configured to become rigid upon positive pressure (e.g., liquid or gas), the catheter 1000 may be depressurized. In this case, the preferred shape of the distal end region 1006 has a straighter shape (compared to the catheter shape in FIG. 10A) such that the distal end region 1006 is pointed toward the target region 1005. Gradually releasing the pressure may allow the catheter 1000 to gently expand and relax to take the turn in the vasculature 1002 with a larger curvature (straighter shape). Gradually changing the pressure can also give the user more control in changing in shape of the catheter 1000 as desired, e.g., without overshooting. During the pressure change, the movement of the distal end region 1006 may be monitoring using any of a number of imaging techniques, such as fluoroscopic (X-ray) imaging, ultrasound (e.g., Doppler) imaging, computed tomography angiography (CTA) imaging, magnetic resonance angiography (MRA) imaging and/or optical coherence tomography (OCT) imaging.

The amount of pressure may be changed until the catheter 1000 takes on a desired shape or is pointed in a desired direction (e.g., as confirmed by imaging). This may mean releasing only some of the rigidizing pressure within the walls of the catheter 1000 or substantially all of the rigidizing pressure within the walls of the catheter 1000. Releasing the pressure may make at least a portion of the catheter 1000 to become more flexible (compared to a rigidized state). The user (e.g., doctor) may control the rate of pressure change, thereby controlling the rate in which the catheter 1000 changes shape. Typically, the change in pressure is at a rate ranging from about 10 mmHg/sec to about 200 mmHg/sec (e.g., between about 20 mmHg/sec to about 150 mmHg/sec, 20 mmHg/sec to about 100 mmHg/sec, 25 mmHg/sec to about 80 mmHg/sec, etc.). In one example, the catheter 1000 may be pressurized to about 6 atmospheres to fully fix it in place, then pressure may be backed down gradually to about 3 atmospheres to allow the catheter 1000 to at least partially relax to a desired shape. Typically, the desired shape may be achieved over a period of seconds (e.g., 1, 5, 10, 20, 30 or 60 seconds).

Once the distal end region 1006 of catheter 1000 takes on the preferred shape, the distal end region 1006 may be near enough to the target region 1005 (e.g., for an aspirating catheter to reach). If so, the catheter 1000 may be rigidized again by the re-application of pressure within the catheter 1000, and the catheter 1000 (e.g., the distal end region 1006) can maintain the desired shape after rigidization.

Sometimes, the position of the catheter 1000 may need further adjustment before re-pressurization and rigidization. For example, while the catheter 1000 is in a more flexible state, the user may pull the catheter 1000 proximally to position the distal end region 1006 and to further change the shape of the distal end region 1006. In some cases, the catheter 1000 may be depressurized, positionally adjusted, and re-pressurized several times. Once the distal end region 1006 is at a desired location, the catheter 1000 may be rigidized so that one or more working tools (e.g., aspirating catheter) may be advanced through the lumen of the rigidizing catheter 1000.

In the example discussed above, the distal end region 1006 of the rigidizing catheter 1000 is configured to take on a straighter shape as pressure (positive or negative) is released from the rigidizing catheter 1000. In other examples, the distal end region 1006 of the rigidizing catheter 1000 may be configured to take on a bent shape as pressure is released from the rigidizing catheter. For example, the distal end region 1006 may have a straighter shape (e.g., as shown in FIG. 10B) when the catheter 1000 is in the rigidized state, and a bent shape (e.g., as shown in FIG. 10A) when the pressure is released and the catheter 1000 is in a more flexible state. This may be accomplished by constructing the distal end region 1006 to have a predetermined bend or bias.

In some cases, the rigidizing catheter 1000 may additionally be steerable by another mechanism other than by relaxation via release of pressure from the walls of the catheter 1000. For example, the catheter 1000 may include one or more steering members, as discussed above. For example, the distal end region 1006 may include one or more mechanical steering members (e.g., one or more tendons, cables, wires, etc., actuators, etc.), pneumatic steering members, magnetic steering members and/or thermal steering members (e.g., using a shape memory alloy or shape memory polymers). Steering the catheter 1000 using the steering members in addition to using the relaxation techniques may allow more refined steering capabilities. For example, in some cases it may be beneficial to use the steering members to point the distal end region 1006 generally a desired direction, then the relaxation technique can be used to refine the position of the distal end region 1006.

In some examples, the rigidizing catheter 1000 does not include any additional steering member or mechanism other than using the relaxation technique via the release of pressure. This may be advantageous in that the catheter 1000 may have a simpler design and steering the catheter 1000 may be less complicated than using a different steering mechanism, or combination of steering mechanisms.

A guidewire may or may not be used during use of any of the rigidizing catheters described herein. For example, in the example of FIGS. 10A and 10B, the catheter 1000 may initially be tracked over a guidewire through at least a portion of the vasculature 602. Then, the guidewire may be removed from the lumen of the catheter 1000. In other cases, no guidewire is used in the advancement and placement of the catheter 1000 within the vasculature 602.

In general, the removal of clot material may result in the loss of large blood volumes. Blood loss can be associated with the duration of the aspiration and the proximity of the aspiration catheter tip to the clot. However, the methods and apparatuses described herein may be configured to minimize blood loss. For example, the use of rigidizing apparatuses as described herein may allow apparatuses to be positioned proximate to the clot, which may minimize the blood volume required to move the clot. The stabilization achieved with the rigidizing catheters described herein may improve access to the clot and lead to better engagement between the tip of the aspiration catheter and the clot, resulting in lower blood loss by virtue of early and improved clot engagement. In addition, the methods and apparatuses described herein may be used without the use of guidewires. In particular, the apparatuses described herein may be configured to accommodate a cardiotomy reservoir (e.g., a suction canister/blood filter), which may be integrated within a thrombectomy circuit as described herein.

As mentioned above, the methods and apparatuses described herein may also improve the steering (e.g., guidance) and operation of suction catheters applied through the rigidizing catheters described herein. For example, the use of dynamic rigidization by the rigidizing catheter provide a sable pathway through the vasculature of the body, and may be used without a guidewire, or with a highly flexible guidewire, where other systems may require the use of guidewire and/or more rigid guidewires. This is because the rigidizing catheter may be frozen and/or locked (or in some cases just partially stiffened) into a shape or pathway that remains relatively fixed. In the stiffer configuration the apparatus may provide a reference platform against which the catheter inserted through the rigidizing catheter (e.g., an aspiration catheter) may be driven. For example, when steering an aspiration catheter, one or more pull wires may be used to change the orientation of the catheter, including to angulate a tip; for the angulation to occur at the distal end of the catheter, the proximal end of the catheter must remain relatively stiff enough to overcome total tension of pull wire. Thus, this may limit the overall flexibility of the catheters used. In contrast the rigidizing catheters described herein may provide the necessary support, when rigidized, to allow for the sufficient mechanical advantage. Thus, a highly flexible aspiration catheter may be used with a rigidizing catheter that may support the proximal end of the more flexible aspiration catheter.

A rigidizing catheter may also act as a stable platform to allow mechanical support for other devices, such as (but not limited to) graspers, imaging system, probes, guidewires, or the like. The distal end of the rigidizing catheter, when rigidized, may act as a control point for such instruments.

Any of the apparatuses described herein may be used in a fully rigidized or partially rigidized configuration. For example, the rigidity of the rigidizing catheters described herein may be adjusted by increasing or decreasing the applied pressure. Typically the higher the pressure (higher positive pressure in some examples), the more rigid the rigidizing catheter can become. This may allow the rigidizing catheter to be partially stiffened. Further, the fac that the rigidizing catheter may be restored to a highly flexible configuration may allow these devices to be inserted and removed without risking damaging the subject's anatomy. In practice, excessively stiff systems typically lead to vascular and hemodynamic complications, and frequently prevent reliable access to anatomies at the end of tortuous pathways. The rigidizing catheters described herein may avoid such concerns by providing an anatomically conformable pathway that does not rely on anatomical support and/or accommodation while greatly extending the reach and stability to anatomies that have been difficult to access. This may allow users (e.g., doctors, technicians, etc.) to better plan their maneuvers, without having to anticipate where the anatomy will be based on catheter movement), and may provide an added level of safety by reducing the urgency that may result from the uncertainty caused by a moving target.

The rigidizing catheters may also protect the adjacent anatomy. Once the rigidizing catheter device is located and rigidized within the body, subsequent instruments introduced through the system are less likely to straighten the access pathway leading to unintended forces on the adjacent anatomy. In its flexible state, the rigidizing catheter typically follows the patient anatomy. In its rigid state, the rigidizing catheter reduces the tendency for inadvertent changes in the pathway. For example, a conventional introducing catheter has a defined flexibility. That sheath may be placed through the heart and the heart may accommodate it. However, when additional instruments are passed through the traditional sheath, the stiffness increases and acts to stiffen the overall system. The resulting straightening can deleteriously affect adjacent anatomical structures, potentially increasing tension on the right ventricle or exacerbating tricuspid valve regurgitation. In contrast, once the rigidizing catheter is placed and rigidized within the anatomy, the ability of any additional instruments to straighten the access pathway is greatly reduced or eliminated. Consequently, the patient remains hemodynamically stable after insertion and rigidizing of the rigidizing catheter, because there will not be further changes in the anatomical pathway while the rigidizing catheter is stabilized, and the anatomy is isolated.

In addition, the rigidizing catheters described herein may allow multiple exchanges with inserted tools without losing position. This allows flexibility and opportunity by freeing the procedure from the requirements for a guidewire, and allows the use of highly “soft” or flexible guidewires (e.g., guidewires having a stiffness of 10 GPa or less, e.g., 9.5 GPa or less, 9 GPa or less, 8.5 GPa or less, 8 GPa or less, 7.5 GPa or less, 7 GPa or less, 6.5 GPa or less, 6 GPa or less, 5.5 GPa or less, 5 GPa or less, 4.5 GPa or less, 4 GPa or less, 4 GPa or less, 3 GPa or less, 2 GPa or less, 1 GPa or less, etc., where stiffness corresponds to the flexural modulus in gigapascals, GPa) than about than may otherwise be possible with other catheters and catheter sheaths. This may improve the safety of the procedure by reducing the potential for perforation or laceration associated with stiff and extra stiff guidewires. Because the rigidizing catheter maintains is position, a role normally relegated to the guidewire, guidewires can be easily exchanged and replaced without loss of position.

Any of the apparatuses described herein may include a pressure control that is configured to control the pressure release rate to allow gradual release of the pressure being used to rigidize the apparatus. For example, the pressure controller may include one or more valves coupled to the flow path within the apparatus, and may be configured to allow the user to selectably release the pressure at a relatively slow rate, as described herein. The pressure controller may include one or more controls (e.g., buttons, sliders, switches, touchscreens, etc.) to activate the gradual release of pressure. The pressure controller may also coordinate the application of the pressure (positive or negative) relatively quickly to rigidize the device, as well as having one or more controls for selectively releasing the pressure at one or more pre-determined and/or user adjustable rates.

The rigidizing catheters described herein may be dynamically rigidized to allow rapid positioning and repositioning. Thus, complex catheter procedures are possible, and the rigidizing catheter may reduce or eliminate ancillary motion due to a stack-up of various flexible systems by stabilizing the entire system in the rigid configuration. This may result in reduced overhead and may simplify endovascular procedures.

### Relative Stiffness

In general, the rigidizing catheters described herein may be transitioned from a highly flexible configuration to a more rigid configuration. In the rigid state, the rigidizing catheter may have a higher stiffness than other aspiration catheters, which may allow for significantly more robust operation, providing a functional advantage of the system over other devices. Localized flexural stiffness measurements on traditional aspiration catheters (e.g., stiffness at localized points along the length of the catheter) as compared to the rigidizing catheter in a rigid configuration have shown that the EI (e.g., Young's modulus x bending moment of inertia), or measurement in units of lbf-inch² show a significant difference. For example, table 1, below shows measured EI values of a 20 F aspiration catheter and a 26 F rigidizing catheter, compared with a commercially available suction catheter (“traditional” catheter, shown as an INARI™ 20 F catheter). The units in table 1 are effective stiffness, EI (lbf-inch²):

TABLE 1

| | | rigidizing catheter | | Asp. | |
|---|---|---|---|---|---|
| | | Flexible | Rigidized | Catheter | Traditional |
| | | state | state | −20 F. | 20 F. |
| without | Proximal | 2.09 | 55.6 | 1.35 | 4.39 |
| obturator | Distal | | | 0.92 | 1.67 |
| With | Proximal | 2.7 | N/A | 1.35 | 6.81 |
| obturator | Distal | | | 0.92 | 2.17 |

The EI stiffness is the stiffness (ratio of bending moment to curvature change) one would get for a homogenous material shaft (like a wire) with a Young's modulus of E and a cross-section corresponding to I. The rigidizing catheter tested are heterogenous in structure, but it is still valid to characterize them with an equivalent EI parameter.

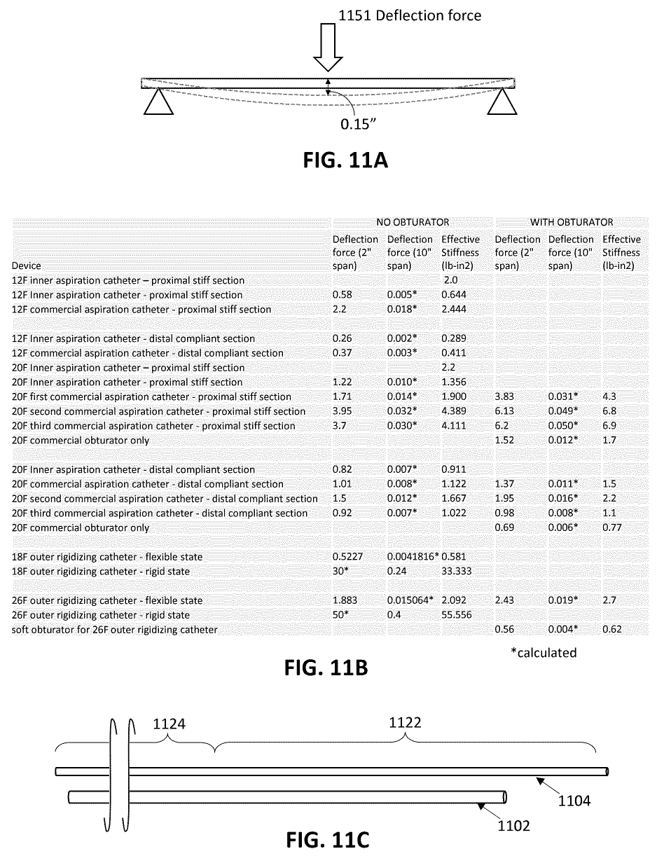

For example, FIG. 11A illustrates one example of an apparatus for determining the deflection force, which is an indicator of (and may be used to estimate) effective stiffness, as shown in the table of FIG. 11B. In FIG. 11A a length of the catheter to be tested is held between two supports and the deflection force 1151 required to deflect the length of catheter a predetermined distance (e.g., 0.15″) may be measured. In some cases the length of the catheter region being tested may be selected, e.g., as a 2-inch span or a 10-inch span. This is also illustrated in the second table, which is shown in FIG. 11B.

Recall the rigidizing catheter may be first delivered up the vessel in its flexible state over its own obturator. Its composite EI stiffness (2.7 lbf-in²) is well below that of the proximal portion of the commercial catheter when it is delivered with its obturator (EI stiffness is 6.81 lbf-in²) and slightly higher that the stiffness of the distal portion of the commercial catheter (EI stiffness is 2.17 lbf-in²). Note the distal portion of the rigidizing catheter is not necessarily intended to reach the target anatomy, so that slight increase of stiffness (2.7 lbf-in² vs 2.17 lbf-in²) isn't really a disadvantage. Once the rigidizing catheter reaches its target location and accommodates to the anatomical curvature, it is rigidized and the obturator is removed after which the EI stiffness becomes 55.6 lbf-in², which is significantly higher than the stiffness of the commercial catheter with its obturator removed. Even though it is 12.7 times stiffer than the commercial catheter (proximal region), it is applying only minimal or negligible compressive tractions to its surroundings in contrast to the commercial catheter which has stored elastic energy needed to keep it in a curved shape and is compressively impinging on its surroundings.

An aspiration catheter may then be delivered up the rigidized rigidizing catheter (on its own obturator, which may be removed after reaching target). The distal portion of the aspiration catheter may protrude beyond the distal end of the rigidizing catheter and traverse the tortuous pulmonary artery anatomy.

The EI stiffness for the distal portion of the Aspiration catheter (0.92 lbf-in²) is significantly lower than the EI stiffness for the distal portion of the commercial catheter (1.67 lbf-in²). This means the aspiration catheter will have an advantage over commercial catheter when accessing tortuous anatomy.

In general, the catheters described herein (e.g., the rigidizing catheter) may be any appropriate length, including up to more than 100 cm (e.g., 110 cm or longer, 115 cm or longer, 120 cm or longer, 125 cm or longer, 130 cm or longer, 135 cm or longer, etc.).

Any of the apparatuses described herein may be configured so that the effective stiffness of the outer rigidizing catheter in the flexible state is within a first range (e.g., less than about 3 lb-inch² or less, less than about 2.5 lb-inch² or less, less than about 2.25 lb-inch² or less, less than about 2.0 lb-inch² or less, less than about 1.75 lb-inch² or less, less than about 1.5 lb-inch² or less, etc.), and the effective stiffness of the outer rigidizing catheter in a rigid configuration, e.g., when positive and/or negative pressure is applied, is greater than about 10 lb-inch² or more (e.g., 12 lb-inch² or more, 14 lb-inch² or more, 16 lb-inch² or more, 18 lb-inch² or more, 20 lb-inch² or more, 25 lb-inch² or more, 30 lb-inch² or more, etc.).

In general, these apparatuses may also include one or more inner aspiration catheters that are configured to extend through and distally out of the outer rigidizing catheter. The inner aspiration catheter may comprise a highly flexible proximal region that extends a large percentage of the length of the inner aspiration catheter (e.g., at least 40% of a length of the inner aspiration catheter, at least 45%, at least 50%, at least 55%, at least 60%, at least 65%, at least 70%, etc.) and may have the same effective bending stiffness of about 2 lb-inch² or less (e.g., 1.5 lb-inch² or less, 1.25 lb-inch² or less, 1.1 lb-inch² or less, 1.0 lb-inch² or less, 0.9 lb-inch² or less, 0.8 lb-inch² or less, 0.7 lb-inch² or less, etc.). In some examples inner aspiration catheter may include a distal, less flexible region having an effective bending stiffness that is greater than about 2 lb-inch².

For example, FIG. 11C shows an example of an outer rigidizing catheter 1102 and an inner aspiration catheter 1104. In this example the distal-most end 1122 (e.g., the distal-most 40%, 45%, 50%, 55%, 60%, etc.) may have a first effective bending stiffness that is, e.g., about 1.5 lb-inch² or less, etc., as described above. The more proximal end region 1124 may have a different effective bending stiffness. The first (distal-most) end region may have essentially the same first effective bending stiffness over the entire length, and the second (proximal) end region may have essentially the same second effective bending stiffness over the entire length. In some cases the transition between the first and second regions and the first and second effective bending stiffness may be abrupt. In some cases it may be beneficial to abruptly transition (e.g., over less than 10 cc, less than 8 cm, less than 7 cm, less than 5 cm, less than 4 cm, less than 3 cm, less than 2 cm, less than 1 cm, etc.) from the highly flexible distal end region (distal region) to the significantly more rigid proximal end region (proximal region). The difference between the effective stiffness of the highly flexible distal region and the more rigid proximal region may be between 1.25× and 100× different (e.g., between about 1.5× and 100×, between about 1.75× and 50×, between about 2× and 50×, between about 2.25× and 40×, or any subregion therebetween). lb-inch².

In general, the apparatuses described herein may take advantage of the stability provided by the outer rigidizing catheter during a procedure. For example, any of these methods may include using the outer rigidizing catheter to minimize or eliminate deviation from the guidewire path when positioning the apparatus. For example, these method may use the outer rigidizing catheter, transitioned to the rigid configuration, to limit or prevent a change in tip position when guidewire is removed. In general, the tip of the catheter may not change position during operation when the outer rigidizing catheter is in the rigid state. For example, the tip angle may change less than x % (e.g., 3%, 4%, 5%, 6%, 7%, 8%, 9%, 10%, 12.5%. 15%, etc.) when the guidewire is removed and/or as the inner catheter (inner aspiration catheter) is moved into and/or out of the outer rigidizing catheter.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of steering a rigidizing device within a body by adjusting a rigidity of the rigidizing device, the method comprising:
maintaining the rigidizing device in a rigid state having a first configuration in a first region of the body by holding a pressure within the rigidizing device at a first pressure;
changing the pressure within the rigidizing device from the first pressure to a second pressure at which the rigidizing device is more flexible than the rigid state, so that the rigidizing device relaxes from the first configuration towards a second configuration;
monitoring movement of the rigidizing device as the rigidizing device relaxes towards the second configuration in a second region of the body; and
rigidizing the rigidizing device by re-applying pressure once the rigidizing device is repositioned within the body in a desired position.

2. The method of claim 1, further comprising repeating the processes of changing the pressure, monitoring movement and rigidizing the rigidizing device to steer the rigidizing device within the body to the second configuration.

3. The method of claim 1, wherein re-applying the pressure comprises applying pressure from the second pressure back towards the first pressure.

4. The method of claim 1, wherein holding the pressure within the rigidizing device at the first pressure comprises holding the pressure against a bladder layer so that the bladder layer within a wall of the rigidizing device drives a rigidizing layer comprising a plurality of filaments against a reinforced layer of the rigidizing device.

5. The method of claim 1, wherein changing the pressure within the rigidizing device comprises leaking the pressure gradually at a rate that is less than the rate that pressure is re-applied to rigidizing the rigidizing device.

6. The method of claim 5, wherein leaking the pressure comprises changing the pressure at a rate of 50 mmHg/sec or less.

7. The method of claim 1, wherein the first configuration comprises a bent or curved configuration.

8. The method of claim 1, wherein the second configuration is set by an elongate member inserted through the rigidizing device.

9. The method of claim 8, wherein the elongate member comprises an obturator.

10. The method of claim 1, wherein the second configuration is set by a shape set on the rigidizing device in the flexible state.

11. The method of claim 1, wherein holding the pressure comprises holding a positive pressure.

12. The method of claim 1, wherein holding the pressure comprises holding a negative pressure.

13. The method of claim 1, wherein the body comprises a branched region within a subject's vasculature.

14. The method of claim 1, further comprising positioning the rigidizing device within the body while the rigidizing device is in the flexible configuration, prior to maintaining the rigidizing device in the rigid state.

15. The method of claim 1, wherein the second configuration of the rigidizing device in the flexible state is a straight configuration.

16. The method of claim 1, wherein changing the pressure comprises releasing the pressure to atmosphere.

17. The method of claim 1, wherein the pressure is hydrostatic pressure.

18. A method of positioning a distal tip region of a rigidizing catheter within a body, the method comprising:
positioning the distal tip region of the rigidizing catheter towards a first vessel while the rigidizing catheter is in a flexible state;
rigidizing the rigidizing catheter from the flexible state to a more rigid state by applying pressure to the rigidizing catheter to hold the distal tip region of the rigidizing catheter oriented towards the first vessel;
changing the pressure in the rigidizing catheter to relax the rigidizing catheter from the rigid state in which the distal tip region of the rigidizing catheter is oriented towards the first vessel into a flexible configuration so that the distal tip region of the rigidizing catheter moves towards a second vessel; and
rigidizing the rigidizing catheter once the distal tip region is oriented towards the second vessel by re-applying pressure within the rigidizing catheter.

19. The method of claim 18, further comprising performing a first procedure with the distal tip region oriented towards the first vessel when the rigidizing catheter is in the rigid configuration and performing a second procedure with the distal tip region oriented towards the second vessel when the rigidizing catheter is in the rigid configuration.

20. The method of claim 19, wherein the first and second procedures comprises removing clot material.

21. The method of claim 18, wherein applying pressure comprises applying positive or negative pressure within a wall of the rigidizing catheter.

22. The method of claim 18, wherein applying pressure comprises applying positive or negative pressure between one or more layers of the rigidizing catheter.

23. The method of claim 18, wherein changing the pressure comprises slowly releasing the pressure.

24. The method of claim 18, wherein changing the pressure comprises repeating the steps of releasing the pressure and re-applying the pressure to orient the distal tip region towards the second vessel before rigidizing the rigidizing catheter once the distal tip region is oriented towards the second vessel.

25. A rigidizing catheter apparatus, the apparatus comprising:
a rigidizing catheter that is configured to transition between a flexible state to a rigid state based on the application of pressure within a wall of the rigidizing catheter; and
a pressure controller that is configured to apply or remove a fluid to control the pressure within the wall of the rigidizing catheter, wherein the pressure controller comprises a first input configured to release pressure from the wall of the rigidizing catheter at a first rate, and a second input configured to release pressure from the wall of the rigidizing catheter at a second rate that is less than the first rate to provide a gradual transition between the rigid and flexible states of the rigidizing catheter.

26. (canceled)

27. The apparatus of claim 25, wherein the pressure controller includes a pressure sensor that is configured to detect the pressure with rigidizing catheter.

28. The apparatus of claim 25, wherein the rigidizing catheter is configured to have a bias shape in the flexible state.

29. The apparatus of claim 28, wherein the bias shape is straight.

30. The apparatus of claim 28, wherein the bias shape is curved.

31. (canceled)

32. (canceled)

33. A system for removing clot, the system comprising:
an outer rigidizing catheter having a flexible state and a rigid state; and
an inner aspiration catheter configured to extend through and distally out of the outer rigidizing catheter, wherein the inner aspiration catheter comprise a highly flexible proximal region that extends at least 40% of a length of the inner aspiration catheter and has an effective bending stiffness of about 1.5 lb-inch2 or less, and wherein the inner aspiration catheter comprises a distal, less flexible region having an effective bending stiffness that is greater than about 2 lb-inch².

34.-61. (canceled)
